(19) United States
(12) Patent Application Publication
(10) Pub. No.: US 2014/0328874 A1
(43) Pub. Date: Nov. 6, 2014

(54) SAMPLE QUANTIFICATION BY DISC CENTRIFUGATION

(71) Applicants: Bernhard Roth, (US); Ame Neumann, (US); NOVARTIS AG, Basel (CH)

(72) Inventors: Bernhard Roth, Lahntal (DE); Arne Neumann, Mockern OT Wormlitz (DE)

(21) Appl. No.: 14/352,246

(22) PCT Filed: Oct. 19, 2012
(86) PCT No.: PCT/IB2012/055756

§ 371 Date: Apr. 16, 2014

Related U.S. Application Data
(60) Provisional application No. 61/627,896, filed on Oct. 19, 2011.

(30) Foreign Application Priority Data
Sep. 10, 2012 (GB) 1216121.2

(51) Int. Cl. G01N 33/569; A61K 39/12

(52) U.S. Cl. CPC G01N 33/56983; A61K 39/12
USPC 424/204.1; 435/5

(57) ABSTRACT

To overcome the limitations of existing particle quantification techniques, the inventors used disc centrifugation in combination with a detector, to quantify particles, in particular virus particles. Also provided is a method for determining particle density using a disc centrifuge. This method is particularly useful for determining virus particle density. Also provided is a method of estimating particle size, based on particle density.

### TECHNICAL FIELD

This invention is in the field of sample quantification, particularly virus quantification.

### BACKGROUND ART

Methods for quantifying viruses are well known in the art. The ability to count and size viruses and their aggregates is becoming increasingly important in virus production and the development of viral vaccines. A problem often encountered in vaccine production is virus aggregation, and it is important to determine the relative concentrations of non-aggregated (monomeric) and aggregated virus material in a sample. Knowing the purity and concentration allows, for example, optimisation of the production process, and a more accurate calculation of virus dosage in the final product.

A number of methods for virus quantification are available, but limitations are associated with each. Moreover, none provides a quick and simple means for quantifying non-aggregated and/or aggregated virus material.

Traditional viral quantification techniques are slow and labour intensive. Infectivity assays, such as virus titration and plaque assays, are standard methods for quantifying viruses which can take up to 3-4 days and require labour-intensive manual counting. Variations on plaque assays, such as fluorescent focus assays, allow for shorter incubation periods, but still require around 24 hours. Another problem with infectivity assays is that they are unable to distinguish between aggregated and non-aggregated viruses. For example, a plaque-forming unit may be from a single virion, or from a single aggregate containing multiple infective viruses. An infectivity assay may therefore underestimate the infectious viral content within a preparation and could not determine the amount of virus particles (infectious and non-infectious).

Some alternative techniques are faster than these traditional techniques, but they are also associated with a number of additional drawbacks. Protein assays, such as the single radial immunodiffusion assay (SRD), or the hemagglutinin assay for example, can offer a faster means for viral quantification, but only provide an indirect measurement of virus concentration, in that they measure proteins, and not virus particles. They are also unable to distinguish between aggregated and non-aggregated viruses. Transmission electron microscopy (TEM) may be used to quantify viruses and also determine the proportion of aggregated and non-aggregated viruses, but its general application is limited by high instrument costs and the amount of space and personnel required for its operation.

Quantitative real time PCR (qRT-PCR) represents the current “gold standard” for virus quantification. While qRT-PCR has the benefit of being able to quantify low-abundance samples, it is relatively slow, typically taking at least 2 hours, particularly for low-abundance samples. PCR-based techniques are also limited in that they measure only target nucleic acids and cannot directly measure virus concentration. In many situations, only the amount of viral protein is relevant, which is not always related to the total amount of virus particles, not the concentration of nucleic acid. Moreover, PCR-based techniques cannot discriminate between intact virions and “free” nucleic acids in solution, which often leads to discrepancies between measurements obtained by qRT-PCR and, for example, plaque assays. Indeed, the ratio of intact virions and free nucleic acids is seldom constant, and so measurements obtained by qRT-PCR may be as variable as the methods by which the samples are obtained. For example, a sample containing multiple disrupted non-viable viruses would give an artificially high result when measured by qRT-PCR. Conversely, virus inactivation (e.g. by beta-propiolactone) can damage virus RNA, which leads to a much lower measured viral concentration. qRT-PCR is also unable to distinguish between aggregated and non-aggregated viruses. Therefore, even the gold standard technique for viral quantification is hampered by numerous and significant limitations.

In view of the above limitations, it is an object of the invention to provide further and improved methods for quantifying non-aggregated and/or aggregated viral material in a sample.

### DISCLOSURE OF THE INVENTION

To overcome the limitations of existing virus quantification techniques, the inventors used disc centrifugation in combination with a detector, to quantify virus particles. Thus, the invention provides a method for quantifying virus particles in a sample using a disc centrifuge.

In one embodiment, the virus sample comprises non-aggregated and/or aggregated virus particles.

In one embodiment, the method comprises the steps of: (a) separating particles in the sample by disc centrifugation (e.g. using a density gradient, preferably a sucrose density gradient); (b) detecting the particles using a particle detector; (c) measuring the particle size distribution; (d) identifying the presence or absence of (i) non-aggregated virus particles, based on the presence of a unimodal size distribution; and/or (ii) aggregated virus particles, based on the presence of a multimodal size distribution; (e) determining (i) the maximum weight of the size distribution and/or (ii) the integrated weight of the size distribution for a sample comprising non-aggregated virus particles; or the integrated weight of the size distribution for a sample comprising aggregated virus particles; and (f) comparing (i) the maximum weight of the size distribution for the sample with the maximum weight of the size distribution of a reference, and/or (ii) comparing the integrated weight of the size distribution for the sample with integrated weight of the size distribution of a reference, and thereby quantifying the virus particles in the sample. The density gradient may further comprise a salt, preferably sodium chloride and/or a buffer, preferably sodium phosphate. In some embodiments, the pH of the separating fluid is between pH3 and 9.

In some embodiments, the sample is treated with a compound with enhanced specificity for biological molecules and which comprises a detectable label. When the detectable label is a fluorescent label, the particle detector is preferably a fluorescence detector.

In some embodiments, the disc centrifuge is a photosedimentometer. In some embodiments, the particle detector uses a detection wavelength in the range of 300-600 nm.

In some embodiments, the virus is an influenza virus.

In some embodiments, the method is used to detect whether whole virus particles were destroyed by detecting a relative increase in the proportion of particle sizes that are smaller than the original virus starting material.

In some embodiments, the method is used to detect the presence or absence of contaminating virus particles in a sample.

In some embodiments, the method is used to detect the presence or absence of contaminating microbe particles in a sample.

In some embodiments, the sample has been treated with a splitting agent. In such cases, the method can determine whether the sample comprises split particles. The method can also be used to determine splitting efficiency of a compound. The method can also be used for determining the susceptibility of a biological particle to splitting.

In some embodiments, the method is for identifying whether antigen particle(s) is/are adsorbed to adjuvant particle(s). The method can be used for identifying the proportion of antigen particles in the sample that is adsorbed to adjuvant particle(s).

For example, the method comprises the steps of: (a) introducing to a sample comprising adjuvant particles and antigen, compound(s) that (i) bind adjuvant particles, (ii) do not bind soluble antigen, and (iii) comprise a detectable label; (b) separating particles in the sample by disc centrifugation; (c) detecting the particles using a particle detector suitable for detecting the detectable label(s); (d) measuring the particle size distribution; (e) identifying the presence or absence of particle size distribution(s) corresponding to (i) adjuvant particles that are not adsorbed to antigen, and/or (ii) adjuvant particles to which antigen is adsorbed; and optionally (f) identifying the proportion of adjuvant particles to which antigen is adsorbed. Preferably, the adjuvant is an aluminium salt.

The method can also comprise the steps of: (a) introducing to a sample comprising adjuvant particles and antigen, compound(s) that (i) bind antigen, (ii) do not bind adjuvant, and (iii) comprise a detectable label; (b) separating particles in the sample by disc centrifugation; (c) detecting the particles using a particle detector suitable for detecting the detectable label(s); (d) measuring the particle size distribution; (e) identifying the presence or absence of particle size distribution(s) corresponding to (i) antigen not adsorbed to adjuvant, and/or (ii) antigen adsorbed to adjuvant particles; and optionally (f) identifying the proportion of antigen adsorbed to adjuvant. Preferably, the compound(s) that (i) bind antigen, (ii) do not bind adjuvant, and (iii) comprise a detectable label, is protein-specific and/or nucleic acid-specific.

In some embodiments, the method is for use in vaccine manufacturing, and the virus particle quantification can be performed in real time.

In another embodiment, the invention provides a process for manufacturing a vaccine comprising the steps of: (i) quantifying non-aggregated and/or aggregated virus particles in a sample taken from a bulk material by using a method of the invention; (ii) optionally adjusting the concentration of the virus particles in the bulk material, based on the quantity of virus particles in the sample, to a concentration that is suitable for use in a vaccine composition; and (iii) preparing the vaccine from the bulk material. The invention also provides a vaccine composition.

Also provided is a method for determining particle density using a disc centrifuge and a method of estimating particle size, based on particle density. In another embodiment, the invention provides a method for determining particle density and/or size using a disc centrifuge comprising the steps of: (a) measuring the settling velocity of a sample in at least two different separating fluids, wherein the fluids have different rheological properties; and (b) performing a regression analysis, preferably a linear regression analysis.

In some embodiments, there is a method for determining particle sedimentation velocity comprising the steps of: measuring the distance from the settling starting point to the detector; determining the retention time; dividing the value obtained from step (a) by the value obtained from step (b).

Methods of the invention are useful for determining e.g. sperm morphologies that manifest in altered density and/or size (see below) using a disc centrifuge, comprising the steps of: (a) measuring the settling velocity of sperm particles in a semen sample in at least two different separating fluids, wherein the fluids have different rheological properties; and (b) performing a regression analysis, preferably a linear regression analysis. Comparing the density and/or size of sperm in a semen sample with a reference (e.g. a textbook reference for typical sperm dimensions and/or densities and/or a reference sample, such as control semen sample) allows identification of atypical sperm density and/or size.

In some embodiments, the method is for use in identifying sperm morphologies that manifest in altered density and/or size. Such a method can comprise the steps of: (i) comparing the density and/or size of sperm in a semen sample with a reference, and (ii) identification of atypical sperm density and/or size in the semen sample.

In some embodiments, there is a method of characterising sperm particles in a semen sample, comprising the steps of: (a) separating particles in the semen sample by disc centrifugation; (b) detecting the particles using a particle detector; (c) measuring the particle size distribution; (d) identifying the presence or absence of i. non-aggregated sperm particles, based on the presence of a unimodal size distribution; and/or ii. aggregated sperm particles, based on the presence of a multimodal size distribution; and/or determining: iii. the maximum weight of the size distribution and/or the integrated weight of the size distribution for a sample comprising non-aggregated sperm particles; or iv. the integrated weight of the size distribution for a sample comprising aggregated sperm particles; comparing v. the maximum weight of the size distribution for the sample with the maximum weight of the size distribution of a reference, or vi. comparing the integrated weight of the size distribution for the sample with integrated weight of the size distribution of a reference and thereby quantifying the virus particles in the sample; and optionally; (A) measuring the sperm particle size sperm particle size variation and/or density variation; (B) comparing the sperm particle size variation and/or density variation with the sperm particle size sperm particle size variation and/or density variation of a reference and thereby identifying the presence or absence of non-homogeneous sperm population in the sample.

In some embodiments, there is a method of identifying sperm morphologies that manifest in altered density and/or size using a disc centrifuge comprising the steps of: a. measuring the settling velocity of sperm particles in a semen sample; b. comparing the settling velocity of sperm particles in the semen sample with the settling velocity of a reference with known settling velocity; c. identifying whether the observed settling velocity of the sperm particles in the semen sample differs from the expected settling velocity of the sperm particles in the semen sample, based on the reference.

In some embodiments, there is a method of identifying abnormal particle size, density and/or quantity in a biological sample using a disc centrifuge comprising the steps of: a. measuring the particle size distribution of a biological sample; b. comparing the particle size distribution of the biological sample with the particle size distribution of a control; c. identifying whether the particle size distribution of the particles in the biological sample differs from the particle size distribution of the control sample; d. determining whether the biological sample contains an abnormally high, low or normal level of particles, compared to the control. The biological sample is preferably a sperm sample, a blood sample or a saliva sample.

Sperm morphologies that manifest in altered density and/or size may also be determined using a disc centrifuge by comparing the settling velocity of sperm particles in a semen sample with the settling velocity of a reference with known settling velocity (e.g. a control semen sample), and identifying whether the observed settling velocity of the sperm particles in the semen sample differs from the expected settling velocity of the sperm particles in the semen sample, based on the reference.

The invention also provides a disc centrifuge for use with methods of the invention.

The Sample

The sample is introduced into the disc centrifuge. Typically, the sample comprises virus particles to be quantified. The concentration of virus particles in the sample is typically unknown. The virus particles may be non-aggregated (monomeric) and/or aggregated. In some embodiments, the sample also comprises cellular components, such as intact cells, cell debris and/or other cell constituents, such as soluble proteins and DNA, in addition to the virus particles. Intact cells and cell debris may be in the cell culture medium after infection and the intracellular content of cells may be released into the medium, e.g. due to spontaneous cell lysis after virus infection. In addition, a sample may contain other materials e.g. cell substrates or their residues, cellular nucleic acids (e.g. DNA), egg proteins, etc. Where the virus is influenza virus, the sample may comprise virus proteins, for example, NS1, PB-1-F2, hemagglutinin, neuraminidase, matrix protein (M1 and/or M2), ribonucleoprotein, nucleoprotein, polymerase complex (PB1, PB2, PA) or subunits thereof, nuclear export protein etc. Virus proteins may be non-aggregated, and/or aggregated. Influenza virus protein aggregates typically comprise matrix protein, e.g. hemagglutinin-matrix protein aggregates etc. The sample may also comprise non-biological particles.

The method of the invention is also useful for detecting and/or quantifying the possible presence of virus particles in a sample e.g. where a sample is suspected of viral contamination. In such cases, the sample may not comprise virus particles i.e. the sample suspected of viral contamination is not contaminated.

The method of the invention is also useful for detecting and/or quantifying the possible presence of microbial contamination in a virus sample, e.g. contaminating bacteria, yeast, fungi etc. Detection of particle diameters of 1 μm or more can indicate microbial contamination of the sample. Also, the size difference between microbial particles and virus particles typically allows determination of virus and optionally microbial particles from a single particle size distribution. Quantification of non-viral particles in a sample may be achieved using the detection methods described herein. Where a sample is suspected of microbial contamination, confirmation of such contamination may be achieved by measuring and comparing particle size distributions of samples taken at different times e.g. at time-points separated by 0.25, 0.5. 0.75, 1, 2, 3, 4, 5, 6, 7, 8, 9, 10, 11, 12, 13, 14, 15, 16, 17, 18, 19, 20, 21, 22, 23, 24, or more hours. Where the sample is contaminated with a growing non-viral microbial population, the particle size distribution obtained from the latter sample will typically show a relative increase in the amount of particles that have diameters of 1 μm or more.

Wherein the sample comprises virus particles, it is preferred that the sample comprises at least 1E07 protected virus copies/ml e.g. approximately 1E08, 1E09, 1E10, 1E11, 1E12, 1E13, 1E14, 1E15, 1E16, 1E17 etc. protected virus copies/ml (i.e. intact virus particles per ml of sample). The number of protected virus copies/ml can be determined using methods known in the art e.g. qRT-PCR etc. Where the sample comprises virus particles, the detection threshold of the method of the invention is approximately 5E09 protected virus copies/ml, and so it is most preferable that the sample comprises at least 5E09 protected virus copies/ml. Therefore, concentration may be useful when there are fewer than e.g. 5E09 protected virus copies/ml.

In some embodiments, the sample is an aliquot of a “bulk material”. In such cases, the concentration of virus in the sample thus represents the concentration in the bulk material. Typically, the volume of the sample is smaller than the volume of the bulk material.

The bulk material typically comprises virus material from virus production e.g. laboratory-scale, or industrial scale virus production. The bulk material may be unprocessed, or may have undergone one or more downstream processing steps e.g. harvest, ultra- and diafiltration, chromatographical purification, density gradient purification etc.

In some embodiments of the invention, the sample comprises other biological particles, for which it is also useful to determine the particle size, density, quantity, etc. Such biological samples include, for example, semen, saliva, urine etc. Such physical information may be used e.g. to determine the biological function (or dysfunction) of such biological particles. Such physical information may also be used to identify whether a biological sample contains e.g. abnormally high or abnormally low particulate levels. “Abnormally high” levels include mere presence of particles that not normally present in the tested biological sample (e.g. bacterial particles in a blood sample or a urine sample). Abnormally high particulate levels in a sample can indicate a disease state e.g. urine casts, which can be indicative of kidney disease. Abnormally low particulate levels in a sample can also indicate a disease state e.g. low reticulocyte levels can indicate anaemia.

When the biological sample is a semen sample, methods of the invention may be used e.g. for fertility studies, by determining whether the sample e.g. has a high, low, or average sperm count, and/or morphologies that manifest in altered density or non-homogeneous sperm (characterised by high sperm particle size variation and/or high density variation) etc. Accordingly, the invention also provides a method for quantifying sperm cells in sample using a disc centrifuge. Sperm cells are approximately 5 μm wide and 50 μm long, and so their particle size distribution is easily distinguishable from e.g. virus particles in a sample (which are considerably smaller). Quantification of sperm particles in a sample may thus performed when the sample also comprises virus particles, or when the sample does not comprise virus particles. Depending on the concentration of particles in the sample, it may be necessary to dilute or concentrate the sample before analysis by disc centrifugation, to ensure accurate measurement. Too high a concentration may cause instability in the sedimentation, and loss of resolution. Samples are typically diluted to under 0.5% solid content, and in many cases to under 0.1% solid content. The required dilution depends upon the average size of the distribution, the refractive index of the particles, and the width (or dispersity) of the particle size distribution. Particle size distribution is typically represented as particle size (x) against particle weight (y). Particle size and/or particle weight may be absolute or relative. Wide particle size distributions, which contain a wide range of particle sizes, require less dilution than narrow distributions, because the particles will spread out during the analysis so that only a small fraction of the total weight of the particles in the sample detected at any given moment. Samples with few and narrow particle distribution narrow peaks may need to be diluted to below 0.1% solid content. This is because the particles in each peak do not spread out as much during centrifugation, and so a large fraction of the total particles in the sample may be in the path of the light beam at one time. Samples comprising particles with higher refractive index usually require more dilution than those comprising particles with a lower refractive index, because they have higher turbidity at equal concentration. In one embodiment, dilution occurs before the sample is introduced to the disc centrifuge e.g. dilution occurs in a syringe that is used to introduce the sample into the disc centrifuge.

Preferably, the diluent is a buffer. The diluent can also be water, or cell growth medium, such as “CDM” (chemically defined medium). Alternatively, the diluent can comprise a combination of two or three of the above. Typical buffers for use as diluents include, for example, phosphate buffered saline, sodium bicarbonate, citrate, tris-buffered saline, amino-methyl-propanediol, citrate-phosphate, diethanolamine, sodium phosphate, tris-buffered saline, sodium citrate, sodium phosphate saline, glycine-HCl, acetic acid, 2-(N-morpholino)ethanesulfonic acid (MES), N-(2-acetamido)-2-aminoethanesulfonic acid (ACES), piperazine-N,N′-bis(2-ethanesulfonic acid) (PIPES), 3-(N-morpholino)propanesulfonic acid (MOPSO), imidazole, 2,2-bis(hydroxymethyl)-2,2′,2″-nitrilotriethanol (BIS-TRIS), 1,3-bis(tris(hydroxymethyl)methylamino)propane (BIS-TRIS propane), N,N-bis(2-hydroxyethyl)-2-aminoethanesulfonic acid (BES), (3-(N-morpholino)propanesulfonic acid MOPS), 4-(2-hydroxyethyl)piperazine-1-ethanesulfonic acid (HEPES), N-[tris(hydroxymethyl)methyl]-2-aminoethanesulfonic acid (TES), 4-(N-morpholino)butanesulfonic acid (MOBS), etc. Preferably the buffer is a phosphate buffer e.g. sodium phosphate, potassium dihydrogen phosphate, disodium phosphate etc. The concentration of each buffer can be optimised according to the sample and is typically between 0 and 2M e.g. approximately 10 mM, 20 mM, 30 mM, 40 mM, 50 mM, 60 mM, 70 mM, 80 mM, 90 mM, 100 mM, 120 mM, 140 mM, 160 mM, 180 mM, 200 mM, 220 mM, 240 mM, 260 mM, 280 mM, 300 mM, etc. Preferably, the buffer concentration is approximately 50 mM, i.e. between 30 and 70 mM, preferably between 40 mM and 60 mM, more preferably between 45 mM and 55 mM.

The pH of the diluent can also be varied to maintain compatibility with the sample. Typically, the pH of the diluent is between pH3 and pH11. Preferably, the pH is between pH5 and pH9, e.g. approximately pH 5, 6, 7, 8, or 9. More preferably, the pH is between pH6 and pH8 (e.g. pH7, pH7.5, or pH8).

Concentration of virus particles in the sample may be achieved using any appropriate method in the prior art, for example, through the use of ultrafiltration membrane, evaporation of the sample etc.

Thus, the sample may, have been concentrated, or diluted, relative to the bulk material. In such cases, the concentration of virus particles in the sample may be used to back-calculate the concentration of virus particles in the bulk material, taking into account dilution or concentration factors.

Virus Aggregation

Virus particles generally exist as monomeric, “non-aggregated” particles. Virus particles may also form aggregates. Aggregates may comprise only virus particles (i.e. two, or more virus particles), or may comprise other material, e.g. cellular components, such as intact cells, cell debris or cell fragments (i.e. one or more virus particle, attached to other material). These are variously referred to as aggregated particles, virus aggregates, aggregated viruses or viral aggregates. The size and mass of virus aggregates is larger than the size and mass of monomeric non-aggregated virus particles.

The method of the present invention can be applied to samples containing aggregated and/or non-aggregated virus particles. The method of the present invention may be used regardless of how the aggregates are formed, either by specific or non-specific interactions. Aggregation may be an intrinsic property of a virus. It may be favoured as a general, non-specific event which occurs under certain circumstances. For example, increased virus concentration within a fluid may lead to the formation of non-specific aggregates due to an increased chance of random collisions between particles. Temperature may also influence virus aggregation.

The presence of certain constituents in a virus-containing fluid may cause virus aggregation. For example, constituents such as zinc or manganese can induce aggregation of influenza virus. Host cell proteins e.g. proteins involved in virus replication and assembly may be released into the medium due to spontaneous cell lysis, and may also be responsible for inducing virus aggregation.

Some steps in virus purification may also promote virus aggregation. For example, nucleases may be used to degrade nucleic acids present in the sample. These enzymes often require elements, such as magnesium, which can induce aggregation. Formaldehyde, for example, is often used to inactivate viruses, but may also cause virus aggregation. Ultrafiltration is frequently used to concentrate a virus early in the purification process and may also lead to increased aggregation.

Disc Centrifugation

Disc centrifugation allows rapid determination of particle size distribution (typically within 3-15 minutes). Disc centrifugation is capable of analysing particles between ˜2 nm and ˜80 μm in diameter, and weighing below 100 ng. It can resolve particles with as little as ˜2% size difference. Disc centrifugation is used in a wide range of applications, for example chemical, pharmaceutical (e.g. particles for drug delivery, diagnostic particles, monitoring of cell disruption, protein clusters, liposomes, micro-encapsulated drugs), semiconductor, printing and painting, nanoparticle development etc. There is no disclosure in the prior art that disc centrifugation could be used to quantify virus particles in a sample. The applicability of disc centrifugation for small particle size distribution has led to its use in determining the presence (but not quantifying) of non-aggregating and/or aggregating virus particles in a sample (for example, see reference 1). A study on nanoparticles (but not virus particles) found that particle size distribution has the potential to yield relative and quantitative data (see reference 2).

The separation of particles by disc centrifugation is based on different sedimentation velocities of particles within the sample. Sedimentation rates differ according to the gravitational field strength (i.e. the rotational speed of the centrifuge disc), the difference in density between the particles, the viscosity of the separating fluid, particle shape and particle size. According to Stokes' law, particles settle in a (separating) fluid under a gravitational field, and sedimentation velocity increases as the square of the particle diameter. Particles sedimenting through the separating fluid are detected as they pass a detector. Therefore, particles with higher size and density have a higher settling velocity and therefore reach the detector earlier than particles with lower size and density. The time between particle injection and detection allows the settling velocity to be calculated, and this settling velocity can be correlated to the size of the particles.

The separating fluid preferably includes a density gradient. Density gradients allow stable sedimentation of particles without streaming effects. A density gradient allows for higher resolution, with separated peaks for each narrow band of particle sizes, rather than generating broad overlapping peaks. Methods of density gradient production are well known in the art, and may be performed manually or automatically.

The density gradient can comprise any suitable combination of soluble materials, such as sucrose, dextran, NaBr, Ficoll (a hydrophilic polysaccharide), cesium salts e.g. CsCl, Cs₂SO₄, CsFormate etc. The density gradient is typically water-based (aqueous), but can be organic e.g. toluene and methyl ethyl ketone (MEK), hydrocarbon oils, polymethyl methacrylate, n-propanol and/or n-butanol. Concentration gradients in the density gradient can vary but can be routinely tailored to optimise separation of particles within the sample e.g. 2-8%, 4-12%, 8-24%, 14-20%, 20-80%, 31-37%, or 51-57% (w/v). The lower concentration limit in the density gradient is typically between 0-10% and the upper concentration limit is typically between 5-30%. There is typically between 2-5 fold difference between the lower and the upper concentration limits. The density of the density gradient at the point of detection is typically lower than the density of the target particles to avoid trapping the target particles within the density gradient.

Water-based gradients typically comprise sucrose and/or dextran, preferably sucrose. Preferably the density gradient is optically clear.

The density gradient optionally further comprises surfactant to help ensure that the particles in the sample do not coagulate during sedimentation. Surfactants can be, for example, anionic, cationic, non-ionic or zwitterionic. The type of surfactant used can depend on the sample. Surfactant may also be present in the sample.

The concentration of surfactant is typically between 0-1% (w/v) e.g. 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.6, 0.7, 0.8, 0.9, 1, or 2% (w/v). Preferably, the concentration of surfactant is 0.02-0.08% (w/v) e.g. 0.05% (w/v).

Anonic surfactants include, for example, sulfate, sulfonate, phosphate, carboxylate, ammonium lauryl sulfate, sodium dodecyl sulfate, sodium lauryl sulfate, sodium myreth sulfate, dioctyl sodium sulfosuccinate, perfluorooctanesulfonate, perfluorobutanesulfonate, alkyl benzene sulfonates, alkyl aryl ether phosphate, alkyl ether phosphate, fatty acid salts, sodium stearate, sodium lauroyl sarcosinate, perfluorononanoate etc.

Cationic surfactants include, for example primary, secondary or tertiary amines, octenidine dihydrochloride, quarternary ammonium cation, cetyl trimethylammonium bromide, cetyl trimethylammonium chloride, cetylpyridinium chloride, polyethoxylated tallow amine, benzalkonium chloride, benzethonium chloride, 5-bromo-5-nitro-1,3-dioxane, dimethyldioctadecylammonium chloride, dimethyldioctadecylammonium bromide etc.

Non-ionic surfactants include, for example, cetyl alcohol, stearoyl alcohol, cetostearoyl alcohol, oleyl alcohol, polyoxyethylene glycol alkyl ethers, octaethylene glycol monododecyl ether, pentaethylene glycol monododecyl ether, polyoxypropylene glycol alkyl ethers, glucoside alkyl ethers, decyl glucoside, lauryl glucoside, octyl glucoside, polyoxyethylene glycol octylphenol ethers, Triton X-100, polyoxyethylene glycol alkylphenol ethers, nonoxynol-9, glycerol alkyl esters, glyceryl laurate, polyoxyethylene glycol sorbitan alkyl esters, polysorbates, sorbitan alkyl esters, cocamide MEA, cocamide DEA, dodecyldimethylamine oxide, block copolymers of polyethylene glycol and polypropylene glycol etc.

Zwitterionic surfactants include, for example, zwittergent 3-10, zwittergent 3-12, zwittergent 3-14, zwittergent 3-16, 3-[(3-cholamidopropyl)dimethylammonio]-1-propanesulfonate, cocamidopropyl hydroxysultaine, amino acids, imino acids, cocamidopropyl betaine, lecithin etc.

There will usually be a difference in refractive index between the particles and the density gradient fluid. If the refractive indexes are exactly the same, and there is no light absorption by the particles, then the particles become “invisible”, since particles with equal refractive index to the density gradient will not scatter light. Most solid materials have a refractive index higher than most fluids, and so this is unlikely to be relevant for methods of the invention.

The disc centrifuge typically comprises an anti-evaporation lid. Optionally, additional agents are used to limit evaporation of the separating fluid e.g. dodecane, tetradecane, mineral oil etc. The use of an anti-evaporation lid and/or the addition of such additional agents can minimise evaporation and deterioration of the separating fluid and so maintain its integrity. The anti-evaporation lid also eliminates drag on the fluid surface from motion of the air relative to the fluid.

The separating fluid optionally further comprises e.g. salts and/or buffer e.g. to minimise degradation, disruption or dehydration of the virus particles.

Where the separating fluid comprises a salt, the salt, or salts, can be varied to maintain compatibility with the sample. As described herein, the addition of salt to the separating fluid can minimise, or prevent virus particle aggregation. Typical salts for use in the separating fluid include, for example, sodium chloride, sodium phosphate, potassium chloride, potassium dihydrogen phosphate, disodium phosphate, and/or magnesium chloride, etc. Other salts may be used in the separating fluid. Where a salt, or salts, are used, sodium chloride is preferred. Salt concentrations may be optimised according to the separating fluid and the sample. The concentration of each salt in the separating fluid is typically between 0 and 1M e.g. 20 mM, 40 mM, 60 mM, 80 mM, 100 mM, 120 mM, 140 mM, 160 mM, 180 mM, 200 mM, 220 mM, 240 mM, 260 mM, 280 mM, 300 mM, etc. Preferably, the salt concentration is between 100 mM and 140 mM, e.g. between 110 mM and 130 mM, preferably between 115 mM and 125 mM, most preferably 120 mM.

Where the separating fluid comprises a buffer, the buffer can also be varied to maintain compatibility with the sample. Typical buffers for use in the separating fluid include, for example, phosphate buffered saline, sodium bicarbonate, citrate, tris-buffered saline, amino-methyl-propanediol, citrate-phosphate, diethanolamine, sodium phosphate, tris-buffered saline, sodium citrate, sodium phosphate saline, glycine-HCl, acetic acid, 2-(N-morpholino)ethanesulfonic acid (MES), N-(2-acetamido)-2-aminoethanesulfonic acid (ACES), piperazine-N,N′-bis(2-ethanesulfonic acid) (PIPES), 3-(N-morpholino)propanesulfonic acid (MOPSO), imidazole, 2,2-bis(hydroxymethyl)-2,2′,2″-nitrilotriethanol (BIS-TRIS), 1,3-bis(tris(hydroxymethyl)methylamino)propane (BIS-TRIS propane), N,N-bis(2-hydroxyethyl)-2-aminoethanesulfonic acid (BES), (3-(N-morpholino)propanesulfonic acid MOPS), 4-(2-hydroxyethyl)piperazine-1-ethanesulfonic acid (HEPES), N-[tris(hydroxymethyl)methyl]-2-aminoethanesulfonic acid (TES), 4-(N-morpholino)butanesulfonic acid (MOBS), etc. Preferably the buffer is a phosphate buffer e.g. sodium phosphate, potassium dihydrogen phosphate, disodium phosphate etc. The concentration of each buffer in the separating fluid is typically between 0 and 2M e.g. approximately 10 mM, 20 mM, 30 mM, 40 mM, 50 mM, 60 mM, 70 mM, 80 mM, 90 mM, 100 mM, 120 mM, 140 mM, 160 mM, 180 mM, 200 mM, 220 mM, 240 mM, 260 mM, 280 mM, 300 mM, etc. Preferably, the buffer concentration is between 30 and 70 mM, more preferably between 40 mM and 60 mM, most preferably between 45 mM and 55 mM, e.g. 50 mM.

The pH of the separating fluid can also be varied to maintain compatibility with the sample. The pH is typically the same throughout the separating fluid. The pH of the separating fluid is typically between pH3 and pH11. Preferably, the pH is between pH5 and pH 9, e.g. pH 5, 6, 7, 8, or 9. More preferably, the pH is between pH6 and pH8 (e.g. pH7, pH 7.5, or pH8). Alternatively, there may be a pH gradient throughout the separating fluid.

Typically, the separating fluid is introduced into a rotating disc. The target rotating speed is the rotating speed to which the disc centrifuge is set. The actual disc rotating speed during a method of the invention can differ from the target rotating speed e.g. if the disc rotating speed is accelerating towards the target rotating speed, or decelerating from the target rotating speed. The target disc rotating speed can be approximately 15000, 16000, 17000, 18000, 19000, 20000, 21000, 21000, 23000, 24000, 25000, 26000, 27000, 28000, 29000, 30000 RPM, or more. This is typically to allow for linearization of the density gradient. The target disc rotation speed may also be varied, although this is generally not preferable. As an alternative, a separating fluid may be introduced into a disc that is not rotating, followed by rotation of the disc at any of the aforementioned target rotation speeds. The separating fluid may be introduced into the disc when the disc is rotating at the target rotating speed, below the target rotating speed, or greater than the target rotating speed. Typically, the separating fluid is rotated for some time prior to the addition of the sample e.g. for approximately 5, 10, 15, 20, 25, 30, 35, 40, 45, 50, 55, or 60 minutes etc. Where the separating fluid comprises a density gradient, this is typically to allow for linearization of the density gradient before introduction of the sample.

The sample is typically inserted into the middle of the disc when the disc is rotating. As an alternative, the sample is inserted into the middle of the disc when the disc is not rotating. As an alternative, the sample is introduced between the middle of the disc and the outer edge of the disc. Some particles in the sample have a lower density than the separating fluid, and so would normally sediment towards the fluid “surface” (i.e. the surface of the disc with the lowest relative density, which is typically the surface of the disc that is nearest to, and also parallel to the axis of rotation) instead of sedimenting towards the “bottom” (i.e. the surface of the disc with the highest relative density, which is typically the surface of the disc that is furthest from, and also parallel to the axis of rotation). A technique has been developed where a low density sample is delivered to the bottom of the centrifuge chamber, rather than to the fluid surface (see reference 3). These low density particles also sediment according to Stokes' law, but in the negative (upward) direction (i.e. towards the fluid surface).

The sample is typically introduced to the disc by injection using e.g. a syringe, although other methods may also be used. The syringe may be e.g. a “Hamilton syringe” (produced by Hamilton Company), a “BD syringe” (produced by Beckton, Dickinson and Company) etc. The introduction of sample to the disc may be automated, e.g. by using an “autosampler” or an “autoinjector” which can be programmed to perform automatic injection of samples into the disc. An autosampler transfers samples from a vial in the autosampler's vial tray. An autoinjector (e.g. produced by CPS Instruments) comprises a carousel which holds a number of syringes (typically 20) that are pre-filled with the sample.

For particle separation, the disc typically rotates at a constant target speed, e.g. approximately 15000, 16000, 17000, 18000, 19000, 20000, 21000, 21000, 23000, 24000, 25000, 26000, 27000, 28000, 29000, 30000 RPM, or more. Higher target disc rotation speed can also be used with the present invention. Target disc rotation speed may be optimised according to the sample. The target disc rotation speed, or speeds, may also be varied during particle separation. The sample may be inserted when the disc is rotating at the target rotation speed, below the target rotation speed, or greater than the target rotation speed. For samples with a broad particle size distribution, the disc can be equipped with special speed ramping inserts (CPS Instruments) that allow the disc speed to be changed during an analysis, without disrupting the density gradient. These speed ramping inserts allow disc speed to be increased (ramped) during an analysis from a low speed (e.g. ˜600 RPM) to a high speed (e.g. ˜24000 RPM), and so increase the practical dynamic range from ˜70 to 1000.

Particles sedimenting through the separating fluid are detected as they pass a detector. Typically, detection of the particles is optical, or uses X-rays. If the detection uses X-rays, it is preferable to use lower-sensitivity X-rays. Optical detection typically involves Mie Theory light scattering corrections. A disc centrifuge which uses optical detection is called a disc centrifuge photosedimentometer. A disc centrifuge that uses X-rays is called an X-ray disc centrifuge.

Particle detection is preferably optical. Detection is preferably performed by light absorption at a certain wavelength, or wavelengths. Preferred particle detection wavelengths for use with the present invention are, in the range of 185-750 nm. Preferably, particle detection wavelengths are between 185-400 nm (e.g. 254), 185-280, 250-290, 280-315, 315-400, 380-750, 380-450 (e.g. 405), 450-475, 476-495, 495-570, 570-590, 590-620, 620-750 nm. This photometric measurement is based on the Beer-Lambert law. The detector is located at a certain distance from the middle of the disc. The concentration of particles at each size is determined by continuously measuring the turbidity of the fluid as it passes the detector i.e. the higher the concentration of particles passing the detector, the more light gets absorbed. The weight of the particles passing the detector may be calculated from the known density of the particles by the application of Mie theory of diffraction of light and Stokes' Law. The weight distribution is converted to a surface area or number distribution, if required.

In some embodiments, detection of particles involves use of compound(s) that bind to the particles, and are more easily detectable than the particles themselves. For example, where the particle is a virus particle, such compounds may bind to e.g. viral DNA and/or viral proteins, and so help detection of the virus particles. Typically, such compounds are used to help improve detection of low concentration(s) of particles in a sample. Also, where a sample contains (a) one or more particle populations of interest (e.g. virus particles), and (b) one or more non-target particle populations that have similar rheological properties to the target particles, but do not bind to the compound(s); then the compound(s) may be used to help detect the target particles within the sample. Such compound(s) are known in the art and typically involve use of e.g. stains or dyes, such as protein-specific antibodies, DNA-specific probes, Hoechst (bisbenzimide) dyes, Quant-iT PicoGreen reagent (Molecular Probes), YO-PRO-1 (Molecular Probes), fluorescein dyes e.g. fluorescein isothiocyanate (FITC), Tetramethylrhodamine isothiocyanate (TRITC), aminomethylcoumarin acetate (AMCA). Typically, such compound(s) includefluorescence label(s), and/or bioluminescence label(s), etc. Such compounds are typically added to the sample before analysis by disc centrifugation, and may require an incubation period before analysis e.g. to permit binding between the biological molecule(s) and the compound(s). Methods of the invention that involve use of compound(s) that help improve detection particles typically also require use of a detector suitable for detecting the compound. For example, where the compound includes a fluorescent label, the detector is preferably a fluorescence detector, preferably a filter fluorometer or a spectrofluorometer. The fluorescence detector may be single-channelled or multi-channelled. Where the invention involves use of a fluorescent label, the invention also typically involves use of an excitation source e.g. laser, photodiode, lamp, xenon arc, mercury-vapor lamp etc. Wavelengths suitable for excitation of fluorophore(s) and concomitant detection of emission may be readily determined.

For example, in some embodiments, the invention involves use of DNA-specific dye(s) e.g. PicoGreen, in combination with a fluorescence detector for the detection of DNA-associated particles, e.g. virus particles. Similarly, in some embodiments, the invention involves use of protein-specific dye(s) e.g. FITC, in combination with a fluorescence detector for the detection of protein-associated particles, e.g. virus particles.

Use of a compound(s) with enhanced specificity for biological molecules and comprising a detectable label, is preferred for methods for detecting and/or quantifying the possible presence of non-viral particles in a sample (e.g. microbial contamination).

Most particle distribution analysis is performed by, but not limited to, proprietary software e.g. the “CPS Software” (CPS Instruments). Particle distribution analysis may performed using non-proprietary software, or by manual or computational analysis of the data.

A disc centrifuge useful for use with methods of the invention is the CPS disc centrifuge (CPS Instruments). Other disc centrifuges may also be used in methods of the invention.

Distinguishing Non-Aggregated and/or Aggregated Virus Particles

A sample comprising singular, non-aggregated particles displays a unimodal size distribution (see, for example FIG. 1). Therefore, the presence of a unimodal size distribution is indicative of the presence of non-aggregated particles.

A sample comprising aggregated particles displays a multimodal size distribution (see, for example FIG. 2). A multimodal sample can comprise 2 or more modes, e.g. 2, 3, 4, 5, 6, 7, 8, 9, 10, or more modes. Therefore, the presence of a multimodal size distribution is indicative of the presence of aggregated virus particles.

Preferably, the size distribution is measured within an approximate size range that is reasonably expected for the monomeric virus particles, and/or aggregates (e.g. virus particles). By measuring the size distribution within the approximate expected virus particle size range, it is possible to minimise the influence of non-target particles (with different sizes) on the virus particle size distribution data. The expected size of the monomeric virus particles, and/or aggregates, may be obtained e.g. experimentally or from the literature. Alternatively, virus particle size may be calculated from the particle density e.g. buoyant density by using methods provided herein. Virus particle sizes may be provided, or obtained e.g. as an average particle size, or as a range of particle sizes.

Methods of the invention allow virus particle quantification based on particle size distribution characteristics. It is not necessary for a sample to comprise only non-aggregated, or only aggregated virus particles. Indeed, an “aggregated” sample comprises non-aggregated particles, but may also comprise non-aggregated particles. Thus, a sample referred to as comprising aggregated particles may comprise 100% aggregated particles, or may comprise greater than e.g. 95%, 90%, 80%, 70%, 60%, 50%, 40% etc aggregated particles. This may be determined by the presence of a unimodal peak in the size distribution, corresponding to the particles, wherein the unimodal peak comprises greater than e.g. 99%, 97%, 95%, 90%, 80%, 70%, 60%, 50%, 40% etc of the particles in the particle distribution.

Methods of the invention are also useful for detecting and/or quantifying the possible presence of aggregated virus proteins in a sample e.g. by detecting the presence or absence of a multimodal size distribution in the estimated size range for aggregates of virus proteins. The size and mass of aggregated virus proteins is typically larger than the size and mass of non-aggregated virus proteins. In one embodiment, aggregates of virus proteins do not comprise virus particles.

The method of the invention is also useful for detecting whether e.g. a processing step, influences aggregation of virus proteins. Aggregation of virus proteins, may be detected e.g. by a relative decrease in the proportion particles with a size and mass expected for monomeric virus proteins and/or an increase in the proportion of particles with a size and mass expected for aggregated virus proteins.

Determining the Maximum Weight of the Size Distribution

The maximum weight (i.e. the maxima) of the size distribution correlates with the amount of mostly non-aggregated particles in the sample. The maximum weight of the size distribution may be determined from e.g. a peak in the particle size distribution data, as indicated by the vertical line on FIG. 1, and also FIGS. 5 and 8 (denoted “maximum”). The maximum weight of the size distribution may be quantified (e.g. by comparison to a reference), or defined in relative terms.

Determining the Integrated Weight of the Size Distribution

The integrated weight of the size distribution correlates with the amount of mostly non-aggregated particles and/or the amount of mostly aggregated particles in the sample e.g. virus particles. The integrated weight of the size distribution may be determined from the size distribution data, as indicated by the shaded area under the curve on FIG. 2, and also FIGS. 5 and 8 (denoted “peak integration”). The integrated weight of the size distribution may be quantified (e.g. by comparison to a reference), or defined in relative terms. Aggregated particles may cause multiple modes, and so it is preferable that the integrated weight is determined within the approximate particle size range to minimise the influence of non-target aggregates. Aggregates can comprise multiple monomeric particles, e.g. multiple virus particles. It is possible to estimate the range of aggregate sizes e.g. by multiplying the expected size of the monomeric particle (e.g. virus particle) by a factor of e.g. 2, 3, 4, 5, 6, 7, 8, 9, 10, or more.

Quantifying the Sample

Quantification may be absolute quantification or relative quantification.

Raw data obtained by a disc centrifuge is usually in the format of time against absorption. It is possible to quantify the virus particles in a sample by comparing its particle size distribution to the size distribution of a reference. A reference has a known concentration of particles and is preferably analysed in the same way as the sample. A reference with a known particle size and particle density may be used to convert analysis time to particle size. Particle size distribution data for the reference is obtainable by any of the methods described herein. Preferably, particles in the reference are physically homogeneous e.g. at least 95% of particles within the reference have a size and/or weight that is ±0-60% of the mean e.g. ±1, 2, 3, 4, 5, 6, 7, 8, 9, 10, 15, 20, 25, 30, 40, 50, 60%.

A reference is typically run before the sample. As an alternative, a reference may be run between samples. As an alternative, a reference may be run after the samples. As an alternative, the reference may be run simultaneously with the sample e.g. reference is added to the sample, such that the sample is internally calibrated. Preferably, a combination of these approaches is used i.e. the reference is run before and between samples, or the reference is run between and after samples, or the reference is run before, between and after samples. It is not necessary to run a reference if the method is pre-calibrated. When setting up the method, it is typically necessary to run a reference so that accurate quantification of virus particles in the sample may be achieved. Where the method has been pre-calibrated, a reference may be run to increase accuracy.

The reference may comprise non-biological and/or biological particles.

Any appropriate non-biological particles may be used in the reference e.g. PVC particles, polystyrene particles, polybutadiene particles, monocrystalline diamond particles, polymethyl methacrylate particles, polyvinylidene chloride particles, PVC homopolymer particles etc.

Any appropriate biological particles may be used in the reference e.g. virus particles. Where the sample comprises virus particles, the type of virus in the reference is preferably same type of virus as in the sample e.g. the reference and the sample both comprise influenza virus particles. Preferably, the reference comprises influenza virus particles that are the same strain as the influenza virus particles in the sample. As an alternative, the reference may comprise two or more virus types and/or virus strains.

The concentration of virus, or viruses, in the reference can be determined by using any one of the virus quantification methods known in the art e.g. infectivity assays, fluorescent focus assays, protein assays, TEM, elisa assays, qRT-PCR etc. Preferably, the virus, or viruses, in the reference is quantified using TEM or qRT-PCR.

Particle size distribution is a function of the concentration of particles, and so it is possible to correlate the maximum weight and/or integrated weight of the particle size distribution with the particle concentration. For non-aggregated particles, the maximum weight and/or integrated weight of the size distribution correlate with particle concentration. For aggregated particles, the integrated weight of the size distribution correlates with particle concentration. By knowing this correlation, it is possible to calculate the concentration of particles in a sample, or samples, which have unknown particle concentration, by comparing the maximum weight(s) and/or integrated weight(s) to that of a reference (i.e. by comparing the size distribution characteristics of the sample(s) which have unknown particle concentration with those of the reference).

A direct correlation between the maximum weight of the size distribution, and also the integrated weight is expected. For the same resolution and proportion of aggregated particles to non-aggregated particles (the size distribution of a different concentrated sample is c f(x), whereby f(x) is the original size distribution and c the concentration factor), maximum weight and integrated weight of the size distribution (total weight) linearity is equivalent due to:

∫ₐ^{b}cf(x)=cF(b)−cF(a)=c(F(B)−F(a))=c∫ₐ^{b}f(x).

Particle sizes and particle densities may also be used to determine the sedimentation velocity of particle populations e.g. by dividing the distance from the settling starting point to the detector by the retention time. Determination of the sedimentation velocity enables the preparative separation of particle populations in e.g. a rate zonal centrifugation, to allow additional analysis without interfering with substances or other particle populations from the sample.

Such methods of the invention are useful for determining e.g. sperm morphologies that manifest in altered density and/or size. Comparing the density and/or size of sperm in a semen sample with a reference (e.g. a textbook reference for typical sperm dimensions and/or densities and/or a reference sample, such as control semen sample) allows identification of atypical sperm density and/or size.

The Virus

The invention is useful for quantifying various types virus, including both enveloped and non-enveloped viruses. It can be used with viruses having a RNA genome (single- or double-stranded) or a DNA genome (single- or double-stranded), and a single-stranded genome may be + or − sense. It can be used with viruses having a segmented genome or a non-segmented genome. It can be used with viruses having a capsid (single or multiple) or viruses having no capsid. Thus the sample may contain one or more of the following:
Orthomyxovirus: The invention may be used to inactive an orthomyxovirus, such as an influenza A, B or C virus. Influenza A or B viruses may be interpandemic (annual/seasonal) strains, or from strains with the potential to cause a pandemic outbreak (i.e., influenza strains with new hemagglutinin compared to a hemagglutinin in currently circulating strains, or influenza strains which are pathogenic in avian subjects and have the potential to be transmitted horizontally in the human population, or influenza strains which are pathogenic to humans). Depending on the particular season and on the nature of the strain, an influenza A virus may be derived from one or more of the following hemagglutinin subtypes: H1, H2, H3, H4, H5, H6, H7, H8, H9, H10, H11, H12, H13, H14, H15 or H16.
Paramyxoviridae viruses: The invention may be used to inactive Paramyxoviridae viruses, such as Pneumoviruses (RSV), Paramyxoviruses (PIV) and Morbilliviruses (Measles).
Pneumovirus: The invention may be used to inactive a Pneumovirus or a metapneumovirus, for example respiratory syncytial virus (RSV), Bovine respiratory syncytial virus, Pneumonia virus of mice, and Turkey rhinotracheitis virus. Preferably, the Pneumovirus is RSV or human metapneumovirus (HMPV).
Paramyxovirus: The invention may be used to inactive a Paramyxovirus, such as Parainfluenza virus (PIV) type 1, 2, 3 or 4, Mumps, Sendai viruses, Simian virus 5, Bovine parainfluenza virus and Newcastle disease virus. Preferably, the Paramyxovirus is PIV or Mumps.
Morbillivirus: The invention may be used to inactive a Morbillivirus, such as Measles.
Picornavirus: The invention may be used to inactive Picornaviruses, such as Enteroviruses, Rhinoviruses, Heparnavirus, Cardioviruses and Aphthoviruses.
Enterovirus: The invention may be used to inactive an Enterovirus, such as Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 and Enterovirus 68 to 71. Preferably, the Enterovirus is poliovirus e.g. a type 1 strain such as Mahoney or Brunenders, a type 2 strain such as MEF-I, or a type 3 strain such as Saukett.
Heparnavirus: The invention may be used to inactive an Heparnavirus (also named Hepatovirus), such as Hepatitis A virus.
Togavirus: The invention may be used to inactive a Togavirus, such as a Rubivirus, an Alphavirus, or an Arterivirus. Rubiviruses, such as Rubella virus, are preferred. Useful alphaviruses for inactivation include aquatic alphaviruses, such as salmon pancreas disease virus and sleeping disease virus.
Flavivirus: The invention may be used to inactive a Flavivirus, such as Tick-borne encephalitis (TBE), Dengue (types 1, 2, 3 or 4), Yellow Fever, Japanese encephalitis, West Nile encephalitis, St. Louis encephalitis, Russian spring-summer encephalitis, Powassan encephalitis.
Hepatitis C virus: The invention may be used to inactive a Hepatitis C virus (HCV).
Pestivirus: The invention may be used to inactive a Pestivirus, such as Bovine viral diarrhea (BVDV), Classical swine fever (CSFV) or Border disease (BDV).
Hepadnavirus: The invention may be used to inactive a Hepadnavirus, such as Hepatitis B virus.
Rhabdovirus: The invention may be used to inactive a Rhabdovirus, such as a Lyssavirus (e.g. a rabies virus) and Vesiculovirus (VSV).
Caliciviridae; The invention may be used to inactive Calciviridae, such as Norwalk virus, and Norwalk-like Viruses, such as Hawaii Virus and Snow Mountain Virus, and Vesivirus, such as Vesicular Exanthema of Swine Virus.
Coronavirus: The invention may be used to inactive a Coronavirus, such as a SARS, Human respiratory coronavirus, Avian infectious bronchitis (IBV), Mouse hepatitis virus (MHV), and Porcine transmissible gastroenteritis virus (TGEV).
Retrovirus: The invention may be used to inactive a Retrovirus, such as an Oncovirus, a Lentivirus or a Spumavirus. An oncovirus may be HTLV-1, HTLV-2 or HTLV-3. A lentivirus may be SIV, HIV-1 or HIV-2.
Reovirus: The invention may be used to inactive a Reovirus, such as an Orthoreovirus, a Rotavirus, an Orbivirus, or a Coltivirus.
Parvovirus: The invention may be used to inactive a Parvovirus, such as Parvovirus B19, or Bocavirus.
Other hepatitis viruses: The invention may be used to inactive a hepatitis delta virus, a hepatitis E virus, or a hepatitis G virus.
Human Herpesvirus: The invention may be used to inactive a Human Herpesvirus, such as Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8).
Papovaviruses: The invention may be used to inactive Papovaviruses, such as Papillomaviruses and Polyomaviruses. Papillomaviruses include HPV serotypes 1, 2, 4, 5, 6, 8, 11, 13, 16, 18, 31, 33, 35, 39, 41, 42, 47, 51, 57, 58, 63 and 65.
Adenoviridae. The invention may be used to inactive adenoviruses, including any of human adenoviruses A, B, C, D, E, F or G.

The virus may be present intentionally (e.g. after growing virus to prepare a vaccine) and/or as a contaminant.

Virus Production

Viruses may be propagated on any suitable substrate e.g. in a cell line culture, in a primary cell culture, in eggs, etc. Cell culture will often use mammalian cells, such as hamster, cattle, primate (including humans and monkeys) and dog cells. Various cell types may be used, such as kidney cells, fibroblasts, retinal cells, lung cells, etc. Examples of suitable hamster cells are the cell lines having the names BHK21 or HKCC. Suitable monkey cells are e.g. African green monkey cells, such as kidney cells as in the Vero cell line (see references 4-6). Suitable dog cells are e.g. kidney cells, as in the CLDK and MDCK cell line. Thus suitable cell lines include, but are not limited to: MDCK; CHO; 293T; BHK; Vero; MRC-5; PER.C6; WI-38; etc. Preferred mammalian cell lines for growing influenza viruses include: MDCK cells (see references 7-10), derived from Madin Darby canine kidney; Vero cells (see references 11-13), derived from African green monkey (Cercopithecus aethiops) kidney; or PER.C6 cells (see reference 14), derived from human embryonic retinoblasts. These cell lines are widely available e.g. from the American Type Cell Culture (ATCC) collection (see reference 15), from the Coriell Cell Repositories (see reference 16), or from the European Collection of Cell Cultures (ECACC). For example, the ATCC supplies various different Vero cells under catalog numbers CCL-81, CCL-81.2, CRL-1586 and CRL-1587, and it supplies MDCK cells under catalog number CCL-34. PER.C6 is available from the ECACC under deposit number 96022940. As well as using mammalian cells, viruses can be grown on avian cells or cell lines (e.g. see references 17-19), including cell lines derived from ducks (e.g. duck retina) or hens e.g. chicken embryo fibroblasts (CEF), etc. Examples include avian embryonic stem cells (see references 17 and 20), including the EBx cell line derived from chicken embryonic stem cells, EB45, EB14, and EB14-074 (see reference 21).

One useful cell line is MDCK (see references 22-24), derived from Madin Darby canine kidney. The original MDCK cell line is available from the ATCC as CCL-34. Derivatives of MDCK cells may also be used. For instance, reference 7 discloses a MDCK cell line that was adapted for growth in suspension culture (‘MDCK 33016’, deposited as DSM ACC 2219). Similarly, reference 25 discloses a MDCK-derived cell line that grows in suspension in serum-free culture (‘B-702’, deposited as FERM BP-7449). Reference 26 discloses non-tumorigenic MDCK cells, including ‘MDCK-S’ (ATCC PTA-6500), ‘MDCK-SF101’ (ATCC PTA-6501), ‘MDCK-SF102’ (ATCC PTA-6502) and ‘MDCK-SF103’ (PTA-6503). Reference 27 discloses MDCK cell lines with high susceptibility to infection, including ‘MDCK.SF1’ cells (ATCC CRL-12042). Any of these MDCK cell lines can be used.

Virus may be grown in suspension culture (e.g. see references 7, 28 & 29) or in adherent culture. One suitable MDCK cell line for suspension culture is MDCK 33016 (deposited as DSM ACC 2219). As an alternative, microcarrier culture can be used.

Viruses may be grown in serum-free culture media and/or protein free media. A medium is referred to as a serum-free medium in the context of the present invention if it contains no additives from serum of human or animal origin. Protein-free is understood to mean cultures in which multiplication of the cells occurs with exclusion of proteins, growth factors, other protein additives and non-serum proteins, but can optionally include exogenous proteins such as trypsin or other proteases that may be necessary for viral growth.

In one embodiment, the method of the invention is used for real time monitoring of virus particle concentration. Real time monitoring may be automated or manual.

The present invention is also useful for determining whether the amount of virus particles in a sample is decreased by a processing step, as indicated by, e.g. a decrease in the amount of virus particles in the sample after a processing step. The amount of virus particles in the sample before and after a processing step may be determined in absolute or relative terms. Some processing steps, especially those involving separation of particles according to their size e.g. filtration, can lead to a decrease in the amount of virus particles in a sample, especially where the sample comprises a distribution of particle sizes. For example, where the cross-sectional area of pores in e.g. a filter is greater than or equal to the cross-sectional area of monomeric particles, but smaller than the cross-sectional area of aggregated particles, then the aggregated particles will not pass through the filter. In such cases, the filtrate will contain fewer virus particles than the feed. Thus, where the amount of virus particles in the sample remains the substantially the same following a processing step e.g. filtration (e.g. at least 85, 87.5, 90, 92.5 95, 96, 97, 98, 99, 99.5 or 100% as many virus particles in the filtrate as the feed), this indicates that the sample does not comprise aggregated virus particles. By contrast, a decrease in the amount of virus particles in a sample following a processing step e.g. filtration, is indicative of the presence of aggregated virus particles in the sample. By comparing the amount of virus particles in a sample before and after a processing step, it is also possible to determine the proportion of monomeric:aggregated virus particles in the sample prior to the processing step. In some embodiments it can be enough merely to compare particle size distributions before and after a processing step: if the processing step removes aggregated virus, but no change in distribution is seen, the conclusion can be that the original sample did not contain aggregated virus.

Virus production and/or vaccine production may also involve one or more aseptic filtration steps, to remove microbial contamination from a sample, e.g. a virus sample. In such cases, the invention is also useful for determining whether such aseptic filtration step(s) have been successful, and to confirm that the sample does not contain a high microbial load (preferably the filtered sample is substantially free of microbial contamination). As discussed above, detection of particle diameters of 1 μm or more typically indicates microbial contamination of the sample. Therefore, absence of a particle size distribution corresponding to particle diameters of 1 μm or more following aseptic filtration indicates that the filtrate does not contain microbial contamination. This method may also be used to determine the efficiency of aseptic filtration step(s).

Vaccines

A vaccine composition may be prepared using live virus, inactivated virus, or by purification of immunogenic protein(s) from a virus e.g. as in the preparation of split or surface antigen vaccines from influenza viruses.

Quantified materials of the invention are ideal for preparing influenza vaccines. Various forms of influenza virus vaccine are currently available (e.g. see chapters 17 & 18 of reference 30) and current vaccines are based either on inactivated or live attenuated viruses. Inactivated vaccines (whole virus, split virion, or surface antigen) are administered by intramuscular or intradermal injection, whereas live vaccines are administered intranasally. The invention can be used with all of these vaccine forms.

Some embodiments of the invention use a surface antigen influenza vaccine (inactivated). Such vaccines contain fewer viral components than a split or whole virion vaccine. They include the surface antigens hemagglutinin and, typically, also neuraminidase. Processes for preparing these proteins in purified form from influenza viruses are well known in the art. The FLUVIRIN™ AGRIPPAL™ and 1NFLUVAC™ products are examples of surface antigen influenza vaccines.

Where the invention uses a surface antigen influenza vaccine, this virus may have been grown in eggs or in cell culture (see above). The current standard method for influenza virus growth for vaccines uses embryonated SPF hen eggs, with virus being purified from the egg contents (allantoic fluid). If egg-based viral growth is used then one or more amino acids may be introduced into the allantoid fluid of the egg together with the virus (see reference 36). Virus is first grown in eggs. It is then harvested from the infected eggs. Virions can be harvested from the allantoic fluid by various methods. For example, a purification process may involve zonal centrifugation using a linear sucrose gradient solution that includes detergent to disrupt the virions. Antigens may then be purified, after optional dilution, by diafiltration. Chemical means for inactivating a virus include treatment with an effective amount of one or more of the following agents: detergents, formaldehyde, β-propiolactone, methylene blue, psoralen, carboxyfullerene (C60), binary ethylamine, acetyl ethyleneimine, or combinations thereof. Non-chemical methods of viral inactivation are known in the art, such as for example UV light or gamma irradiation.

Methods of the invention may be used to determine the effect of virus inactivating agents on virus aggregation e.g. by comparing the particle size distributions of the virus sample before and after administration of the virus inactivating agent. It is useful to know the effect of a virus inactivating agent on virus particle aggregation because particle aggregation induced during or after inactivation can lead to virus loss following e.g. post-inactivation filtration. Virus inactivating agents that do not induce particle aggregation are typically preferred.

When inactivating virus particles, it is also useful to determine whether a sample comprises aggregates of virus particles. This may be achieved by methods of the invention as described herein. A sample comprising virus particle aggregate(s) may be less susceptible to inactivation since the virus inactivating agent may not be able to penetrate the aggregate and inactivate the inner virus particles. In such cases, there is a risk that the sample still contains live virus particles, which may not be desirable. Therefore, if a sample comprises aggregated virus particles, it may be necessary to e.g. use a higher concentration of inactivation agent, use a more aggressive inactivation agent, administer the inactivating agent for a longer time etc. Some embodiments of the invention can use whole virus, split virus, virosomes, live attenuated virus, or recombinant hemagglutinin. These vaccines can easily be distinguished from surface antigen vaccines by testing their antigens e.g. for the presence of extra influenza virus proteins.

Whole inactivated virus can be obtained by harvesting virions from virus-containing fluids (e.g. obtained from eggs or from culture medium) and then treating them as described above.

Split virions are obtained by treating purified virions with detergents (e.g. ethyl ether, polysorbate 80, deoxycholate, tri-N-butyl phosphate, Triton X-100, Triton N101, cetyltrimethylammonium bromide, Tergitol NP9, etc.) to produce subvirion preparations, including the ‘Tween-ether’ splitting process. Methods of splitting influenza viruses, for example are well known in the art e.g. see references 31-36, etc. Splitting of the virus is typically carried out by disrupting or fragmenting whole virus, whether infectious or non-infectious with a disrupting concentration of a splitting agent. The disruption results in a full or partial solubilisation of the virus proteins, altering the integrity of the virus. Preferred splitting agents are non-ionic and ionic (e.g. cationic) surfactants e.g. alkylglycosides, alkylthioglycosides, acyl sugars, sulphobetaines, betains, polyoxyethylene-alkylethers, N,N-dialkyl-Glucamides, Hecameg, alkylphenoxy-polyethoxyethanols, NP9, quaternary ammonium compounds, sarcosyl, CTABs (cetyl trimethyl ammonium bromides), tri-N-butyl phosphate, myristyltrimethylammonium salts, lipofectin, lipofectamine, and DOT-MA, the octyl- or nonylphenoxy polyoxyethanols (e.g. the Triton surfactants, such as Triton X-100 or Triton N101), polyoxyethylene sorbitan esters (the Tween surfactants), polyoxyethylene ethers, polyoxyethlene esters, etc. One useful splitting procedure uses the consecutive effects of sodium deoxycholate and formaldehyde, and splitting can take place during initial virion purification (e.g. in a sucrose density gradient solution). Thus a splitting process can involve clarification of the virion-containing material (to remove non-virion material), concentration of the harvested virions (e.g. using an adsorption method, such as CaHPO₄ adsorption), separation of whole virions from non-virion material, splitting of virions using a splitting agent in a density gradient centrifugation step (e.g. using a sucrose gradient that contains a splitting agent such as sodium deoxycholate), and then filtration (e.g. ultrafiltration) to remove undesired materials. Split virions can usefully be resuspended in sodium phosphate-buffered isotonic sodium chloride solution. Examples of split vaccines are the BEGRIVAC™, INTANZA™, FLUARIX™, FLUZONE™ and FLUSHIELD™ products.

Virosomes are nucleic acid free viral-like liposomal particles (see reference 37). They can be prepared by solubilization of virus with a detergent followed by removal of the nucleocapsid and reconstitution of the membrane containing the viral glycoproteins. An alternative method for preparing virosomes involves adding viral membrane glycoproteins to excess amounts of phospholipids, to give liposomes with viral proteins in their membrane.

Live attenuated viruses are obtained from viruses (grown in eggs or in cell culture), but the viruses are not inactivated. Rather, the virus is attenuated (“att”) e.g. so as not to produce influenza-like illness in a ferret model of human influenza infection. It may also be a cold-adapted (“ca”) strain i.e. it can replicate efficiently at 25° C., a temperature that is restrictive for replication of many wildtype influenza viruses. It may also be temperature-sensitive (“ts”) i.e. its replication is restricted at temperatures at which many wild-type influenza viruses grow efficiently (37-39° C.). The cumulative effect of the ca, ts, and att phenotype is that the virus in the attenuated vaccine can replicate in the nasopharynx to induce protective immunity in a typical human patient, but it does not cause disease i.e. it is safe for general administration to the target human population. These viruses can be prepared by purifying virions from virion-containing fluids e.g. after clarification of the fluids by centrifugation, then stabilization with buffer (e.g. containing sucrose, potassium phosphate, and monosodium glutamate). Live vaccines include the FLUMIST™ product.

Vaccine compositions usually include components in addition to their antigens e.g. they typically include one or more pharmaceutical carrier(s) and/or excipient(s). A thorough discussion of such components is available in reference 38. A vaccine composition may also include an adjuvant e.g. as disclosed in references 39 and 40 (for example, an adjuvant comprising one or more aluminium salts, or comprising a submicron oil-in-water emulsion).

Vaccine compositions are preferably in aqueous form, particularly at the point of administration, but they can also be presented in non-aqueous liquid forms or in dried forms e.g. as gelatin capsules, or as lyophilisates, etc.

Vaccine compositions may include one or more preservatives, such as thiomersal or 2-phenoxyethanol. Mercury-free compositions are preferred, and preservative-free vaccines can be prepared.

Vaccine compositions can include a physiological salt, such as a sodium salt e.g. to control tonicity. Sodium chloride (NaCl) is typical, which may be present at between 1 and 20 mg/ml. Other salts that may be present include potassium chloride, potassium dihydrogen phosphate, disodium phosphate dehydrate, magnesium chloride, calcium chloride, etc.

Vaccine compositions can have an osmolality of between 200 mOsm/kg and 400 mOsm/kg, e.g. between 240-360 mOsm/kg, or between 290-310 mOsm/kg.

Vaccine compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

Vaccine compositions typically have a pH between 5.0 and 9.5 e.g. between 6.0 and 8.0.

Vaccine compositions are preferably sterile.

Vaccine compositions preferably non-pyrogenic e.g. containing <1 EU (endotoxin unit, a standard measure) per dose, and preferably <0.1 EU per dose.

Vaccine compositions are preferably gluten free.

Vaccine compositions may include detergent e.g. a polyoxyethylene sorbitan ester surfactant (known as ‘Tweens’), an octoxynol (such as octoxynol-9 (Triton X-100) or t-octylphenoxypolyethoxyethanol), a cetyl trimethyl ammonium bromide (‘CTAB’), or sodium deoxycholate. The detergent may be present only at trace amounts.

A vaccine composition may include material for a single administration, or may include material for multiple immunizations (i.e. a ‘multidose’ kit). The inclusion of a preservative is useful in multidose arrangements. As an alternative (or in addition) to including a preservative in multidose compositions, the compositions may be contained in a container having an aseptic adaptor for removal of material.

Vaccine compositions are typically administered in a dosage volume of about 0.5 ml, although a half dose (i.e. about 0.25 ml) may be administered to children.

Vaccine compositions can be administered in various ways. The most preferred route is by intramuscular injection (e.g. into the arm or leg), but other available routes include subcutaneous injection, intranasal, oral, intradermal, transcutaneous, transdermal, etc.

Vaccine compositions are suitable for administration to animal (and, in particular, human) patients, and thus include both human and veterinary uses. They may be used in a method of raising an immune response in a patient, comprising the step of administering the composition to the patient.

The immune response raised by these methods will generally include an antibody response, preferably a protective antibody response. Methods for assessing antibody responses, neutralizing capability and protection after viral vaccination are well known in the art. For influenza virus, for instance, human studies have shown that antibody titers against HA are correlated with protection.

As mentioned above, a vaccine composition can include one or more adjuvant(s), which can function to enhance the immune responses (humoral and/or cellular) elicited in a patient who receives the composition. A useful adjuvant can comprise one or more aluminium salts. Another useful adjuvant can comprise an oil-in-water emulsion. Other useful adjuvants are known in the art.

The adjuvants known as aluminum hydroxide and aluminum phosphate may be used, singly or in combination. These names are conventional, but are used for convenience only, as neither is a precise description of the actual chemical compound which is present (e.g. see chapter 9 of reference 41). The invention can use any of the “hydroxide” or “phosphate” adjuvants that are in general use as adjuvants. The concentration of Al⁺⁺⁺ in a composition for administration to a patient is preferably less than 5 mg/ml e.g. ≦4 mg/ml, ≦3 mg/ml, ≦2 mg/ml, ≦1 mg/ml, etc. A preferred range is between 0.3 and 1 mg/ml. A maximum of 0.85 mg/dose is preferred.

Various useful oil-in-water emulsion adjuvants are known, and they typically include at least one oil and at least one surfactant, with the oil(s) and surfactant(s) being biodegradable (metabolisable) and biocompatible. The oil droplets in the emulsion are generally less than 1 μm in diameter, with these small sizes being achieved with a microfluidiser to provide stable emulsions. Droplets with an average diameter which is <220 nm are preferred as they can be subjected to filter sterilisation. Useful adjuvants can include squalene and/or polysorbate 80. Suitable adjuvants which can be used include those known as MF59 and AS03.

Vaccine compositions can be administered by a single dose schedule or a multiple dose schedule. Multiple doses may be used in a primary immunization schedule and/or in a booster immunization schedule. In a multiple dose schedule the various doses may be given by the same or different routes e.g. a parenteral prime and mucosal boost, a mucosal prime and parenteral boost, etc. Administration of more than one dose (typically two doses) is particularly useful in immunologically naïve patients. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Methods of the invention are useful for determining whether and optionally by how much a biological sample (e.g. a virus sample) has been split e.g. following a change in the particle size distribution of the particles before and after splitting. Methods of the invention may also be used to determine the splitting efficiency of a splitting agent and/or the susceptibility of the biological particle to splitting. Methods of splitting biological particles are well known, and are briefly discussed above. For example, complete splitting is detected by the absence of a particle peak corresponding to a biological particle, e.g. virus particles. Partial and complete splitting can be detected by a decrease in the particle peak corresponding to a biological particle, e.g. virus particles. Biological particles that have not been split are identified by no or minimal change to the particle peak following treatment with the splitting agent.

Methods of the invention are also useful for detection, characterisation and quantification of adjuvant particles in a sample, particularly for insoluble adjuvant particles e.g. aluminium salt particles.

Methods of the invention are useful for identifying the proportion of antigen that is adsorbed to adjuvant particles (e.g. aluminium salt). This may be achieved e.g. by comparison of the particle size distributions of antigen, adjuvant particles, and particles comprising antigen adsorbed to adjuvant (such adsorbed adjuvant particles are typically larger adjuvant particles to which antigen is not adsorbed). Typically, adsorption of the antigen to adjuvant leads to (i) decreased proportion of particles corresponding to adjuvant only and (ii) increased proportion of particles corresponding to antigen adsorbed to adjuvant.

Such identification may involve use of compound(s) that bind the adjuvant particles, but do not bind soluble antigen, and which are more easily detected than the adjuvant particles themselves (e.g. via a detectable label). The detectable label(s) thus “decorate” the adjuvant particles, to which antigen particles may be adsorbed. Identifying whether (a) antigen is/are adsorbed to adjuvant particles, and/or (b) what proportion of antigen in the sample is adsorbed to adjuvant particles may comprise use of e.g. (i) adjuvant, preferably aluminium salt adjuvant; (ii) antigen; (iii) compound that binds adjuvant but does not bind soluble antigen, and comprise(s) a detectable label; and detection of the detectable label(s). For example, the method may comprise the steps of (a) introducing to a sample comprising adjuvant particles and antigen a compound that binds adjuvant but does not bind soluble antigen, and comprises a detectable label; (b) separating particles in the sample by disc centrifugation; (c) detecting the particles using a particle detector suitable for detecting the detectable label(s); (d) measuring the particle size distribution; (e) identifying the presence or absence of particle size distribution(s) corresponding to (i) adjuvant particles and/or (ii) antigen adsorbed to adjuvant particles; and (f) identifying the proportion of adjuvant to which antigen is adsorbed.

Identification may also involve use of compound(s) that bind antigen (e.g. using detectable label(s), such as protein-specific and/or DNA-specific reagent(s)), but do not bind adjuvant. The detectable label(s) thus “decorate” the antigen, which may be adsorbed to adjuvant. Identifying whether (a) antigen is/are adsorbed to adjuvant particles, and/or (b) what proportion of antigen in the sample is adsorbed to adjuvant particles, may comprise use of e.g. (i) adjuvant, preferably aluminium salt adjuvant; (ii) antigen; (iii) compound that binds antigen, but does not bind adjuvant, and comprises a detectable label; and detection of the detectable label(s). For example, the method may comprise the steps of (a) introducing to a sample comprising antigen and adjuvant particles a compound that binds antigen but not adjuvant, and comprises a detectable label; (b) separating particles in the sample by disc centrifugation; (c) detecting the detectable label(s) using a suitable particle detector; (d) measuring the particle size distribution; (e) identifying the presence or absence of particle size distribution(s) corresponding to labelled antigen adsorbed to adjuvant; and optionally (f) identifying the proportion of antigen adsorbed to adjuvant.

Such identification may also involve use of (a) compound(s) that bind the adjuvant particles, but do not bind soluble antigen, and comprise a detectable label, and (b) compound(s) that bind antigen (e.g. using detectable label(s), such as protein-specific and/or DNA-specific reagent(s)), but do not bind adjuvant, followed by the steps outlined above. In such cases, the compound that binds the antigen and the compound that binds the adjuvant particles preferably comprise a different detectable label e.g. so that detection may be performed on the same sample, preferably simultaneously using detectors suitable for detecting the respective detectable labels. Adsorption of antigen to adjuvant particles is typically identifiable by co-localisation of the detectable labels on the particle size distribution.

Methods of the invention are also useful for optimising adjuvant particle production. Methods of the invention are useful for determining whether a production method (e.g. for producing virus particles and/or adjuvant particle(s)) produces intended or desired particle size(s).

### BRIEF DESCRIPTION OF DRAWINGS

FIG. 1 shows the unimodal size distribution for non-aggregating influenza virus (influenza strain H3N2).

FIG. 2 shows the multimodal size distribution for aggregating influenza virus (influenza strain H1N1).

FIG. 3 shows the integrated weight of the size distribution against varying concentrations of 263 nm PVC particles in water. A high correlation coefficient R² indicates a linear correlation between injected particle concentration and integrated weight of the size distribution. Measurements were performed in triplicates. Error bars indicate standard deviation.

FIG. 4 shows the maximum weight of the size distribution against varying concentrations of 263 nm PVC particles in water. A high correlation coefficient R² indicates a linear correlation between injected particle concentration and maximum weight of the size distribution. Measurements were performed in triplicates. Error bars indicate standard deviation.

FIG. 5 shows size distributions of the PVC particles tested in FIGS. 3 and 4 at different dilutions. Measurements were performed using a 2-8% (w/v) sucrose gradient in 50 mM NaPO₄, pH 7.5.

FIG. 6 shows the integrated weight of the size distribution against the concentration of influenza H3N2 particles in water. A high correlation coefficient R² indicates a linear correlation between injected particle concentration and integrated weight of the size distribution. Measurements were performed in triplicates. Error bars indicate standard deviation.

FIG. 7 shows the maximum weight of the size distribution against the concentration of influenza H3N2 particles in water. High correlation coefficient R² indicates a linear correlation between particle concentration injected into the disc and maximum weight of the size distribution. Measurements were performed in triplicates. Error bars indicate standard deviation.

FIG. 8 shows representative size distributions of the H3N2 influenza virus particles tested in FIGS. 6 and 7 at different dilutions. Measurements were performed using a 2-8% (w/v) sucrose gradient in 50 mM NaPO₄, pH 7.5.

FIG. 9 shows a summary of the downstream processes that were used for the production of tested influenza particles.

FIG. 10 shows the correlation between the maximum weight of the size distribution (denoted maxima) against the number of injected protected copies per ml and hemagglutinin levels for different H3N2 samples that were purified using the downstream processes summarised in FIG. 9.

FIG. 11 shows the correlation between the integrated weight of the size distribution against the number of injected protected copies per ml and hemagglutinin levels for different H3N2 samples that were purified using the downstream processes summarised in FIG. 9.

FIG. 12 shows the correlation between the maximum weight of the size distribution (denoted maxima) against the number of protected copies per ml and hemagglutinin levels for different H1N1 samples that were purified using the downstream processes summarised in FIG. 9. The H1N1 strain showed aggregation for some buffers, and the linear correlation between the maximum weight of the size distribution and the concentration of injected particles is reduced upon aggregation.

FIG. 13 shows the correlation between the integrated weight of the size distribution (denoted peak integration) against the number of protected copies per ml and hemagglutinin levels for different H1N1 samples that were purified using the downstream processes summarised in FIG. 9.

FIG. 14 shows the correlation between the maximum weight of the size distribution (denoted maxima) against the number of protected copies per ml and hemagglutinin levels for samples of a B strain of influenza that were purified using the downstream processes summarised in FIG. 9.

FIG. 15 shows the correlation between the integrated weight of the size distribution (denoted peak integration) against the number of protected copies per ml and hemagglutinin levels for samples of a B strain of influenza that were purified using the downstream processes summarised in FIG. 9.

FIG. 16 shows the correlation between maximum weight of the size distribution (denoted maxima) against the number of protected copies per ml for all three tested strains of influenza that were purified using the downstream processes summarised in FIG. 9.

FIG. 17 shows the correlation between the integrated weight of the size distribution (denoted peak integration) against the number of protected copies per ml for all three tested strains of influenza that were purified using the downstream processes summarised in FIG. 9.

FIG. 18 shows the aggregation influence of pure water used as gradient density fluid against physiological buffer (50 mM NaPO₄ pH7.5).

FIG. 19 shows the influence of low pH density gradient on size distribution of influenza samples (50 mM NaPO₄ pH 7.5 against 50 mM NaPO₄ pH5.7).

FIG. 20 shows the influence of salt in the density gradient on size distribution of influenza samples of the H3N2 influenza strain (50 mM NaPO₄ pH7.5, against 120 mM NaCl, 50 mM NaPO₄, pH7.5).

FIG. 21 shows disaggregation of influenza aggregates by 1500 bar high pressure homogenization versus original material of the aggregated influenza strain.

FIG. 22 shows size distribution of the H1N1 influenza strain. The curves relate to original material and beta-propiolactone-inactivated material. Disc centrifuge shows only a loss of 7% by quantification using the maximum weight of the size distribution, and 8% by quantification over the integrated weight distribution of the size distribution.

FIG. 23 shows size distributions of the H3N2 influenza strain, produced in MDCK cells at different times after infection: 24 h, 42 h, 48 h and 69 h.

FIG. 24 shows an example of two particle solutions (influenza particles and PVC particles) settling through fluids of different viscosity and density. For a higher density and viscosity, particles have a longer settling time due to lower gravitational force (due to a higher density of the fluid and a higher drag force (due to the higher viscosity).

FIG. 25 shows a simulation of settling time over fluid density with particles of density of 1.385 g/mL for different fluids with different densities but constant viscosities.

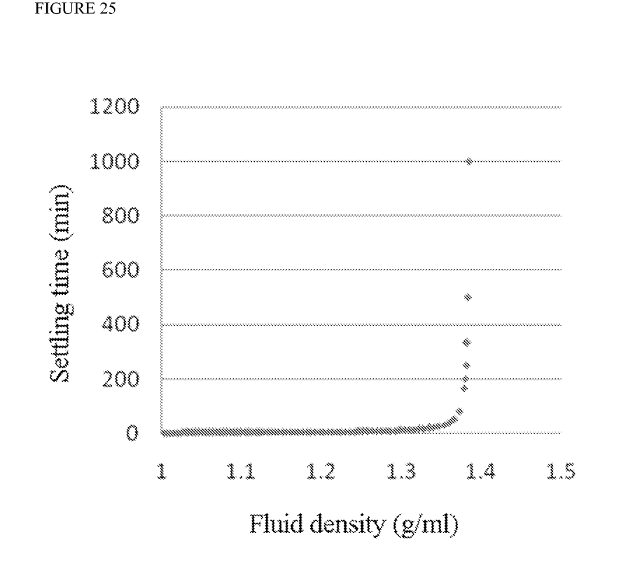

FIG. 26 demonstrates that reciprocal transformation of settling time over fluid density leads to a linear correlation.

FIG. 27 shows a regression analysis for the estimation of the particle density of tomato mosaic virus.

FIG. 28 shows a regression analysis for the estimation of particle density of PVC particles. All measurements were done in triplicate, error bars indicate the standard deviation. The intersection of the regression line with the abscissa corresponds to the particle density.

FIG. 29 shows detection of microbial contamination (at >1 μm) before (curve A) and after (curve B) aseptic filtration.

FIG. 30 shows an example of a complete splitting step—no remaining whole particles could be detected after splitting (curve A) compared to the sample before splitting, containing whole virus particles (curve B).

FIG. 31 shows an example of an incomplete splitting step. In contrast to FIG. 30, less split agent was used (curves A and B), resulting in an incomplete splitting, as remaining whole virus particles could be detected. Curve C shows the un-split native sample (control).

FIG. 32 shows an example of measured adjuvant particles (aluminium hydroxide and aluminium phosphate), each of which has a hydrodynamic diameter of approximately 6 μm.

### MODES FOR CARRYING OUT THE INVENTION

### Linearity and Reproducibility of PVC Standards

To determine whether particle size distribution data obtained by disc centrifugation could be used to calculate particle concentration, the inventors first performed a study using certified PVC standard particles (CPS instruments) with known size and density. 263 nm and 225 nm PVC standard particles were chosen because they are approximately the same size as the virus particles used in subsequent experiments.

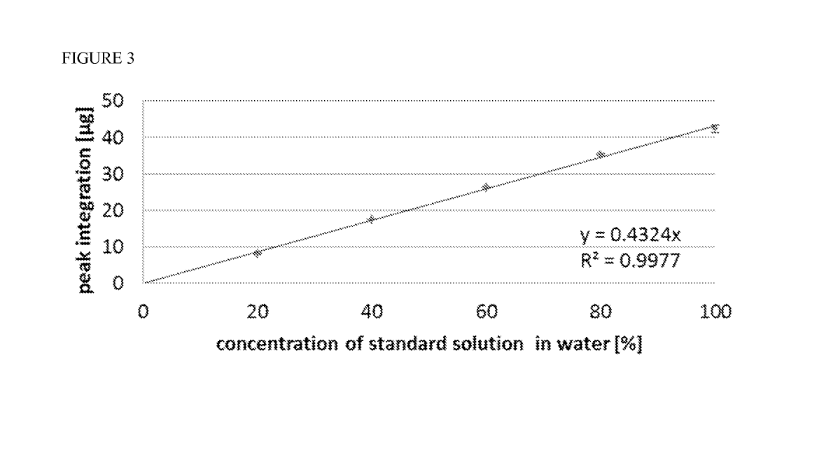

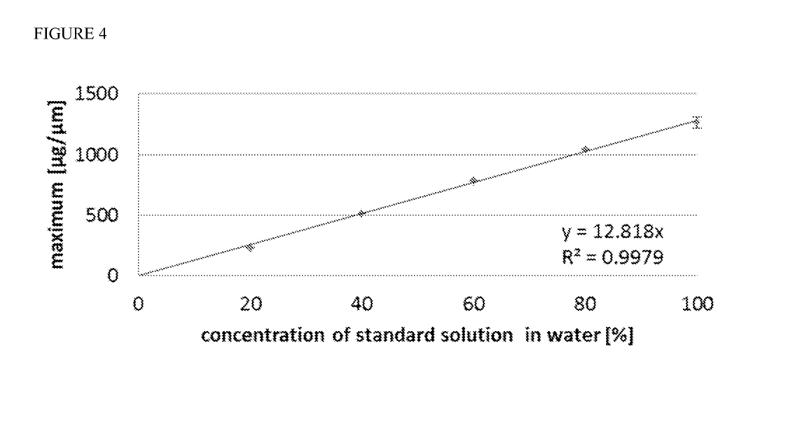

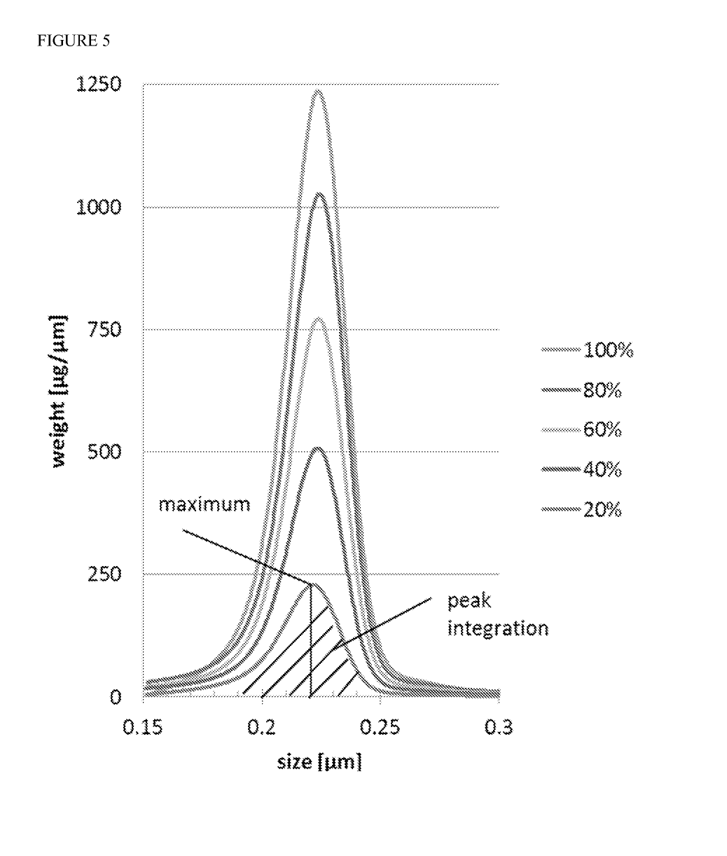

The PVC particles were diluted in water to 80%, 60%, 40% and 20% (v/v) of the starting material. Undiluted PVC starting material was also used. All measurements were performed in triplicate to test for reproducibility. As indicated by the standard deviation of the measurements, this method proves highly reproducible (see FIGS. 3-5).

Operation of the Disc Centrifuge

The disc centrifuge used in the present study was manufactured by CPS instruments (model 24000UHR). Density gradient solutions were prepared manually using a 2% (w/v) sucrose solution and a 8% (w/v) sucrose solution, diluted in 50 mM NaPO₄, pH7.5 and 50 mM NaPO₄, pH7.5, 120 mM NaCl, respectively. These solutions were then mixed to obtain 9 sucrose solutions, containing 2, 2.75, 3.5, 4.25, 5, 5.75, 6.5, 7.25, 8% (w/v). The disc centrifuge was set to 24000 RPM. After reaching 24000 RPM and applying the anti-evaporation lid, 1.6 ml of each sucrose solution was injected into the disc, beginning with the 8% (w/v) solution, ending with the 2% (w/v) solutions. To further prevent evaporation, and to prolong stability 0.5 ml of dodecane was applied onto the established density gradient. After 20-30 min run time to allow for linearization of the density gradient, 2000 μL of sample was injected using a syringe for precise volume injection. The measuring parameters were: particle density 1.16 g/ml, maximum size 2 μm, minimum size 0.04 μm, particle refractive index 1.54, particle absorption 0.001.

Results

The inventors identified two features of the PVC size distribution that correlate with the particle concentration: the maximum weight of the size distribution (i.e. the maxima) and the integrated weight of the size distribution. Results are shown in FIGS. 3-5. Both the integrated weight and the maximum weight of the size distribution showed a linear correlation to the concentration of the injected PVC particles. This is indicated by high correlation coefficients between the injected concentration of PVC particles and the maximum weight and integrated weight of the size distribution (0.9979 and 0.9977, respectively). All values were highly reproducible with a maximal standard deviation of less than 5%.

Linearity and Reproducibility of a Virus Sample

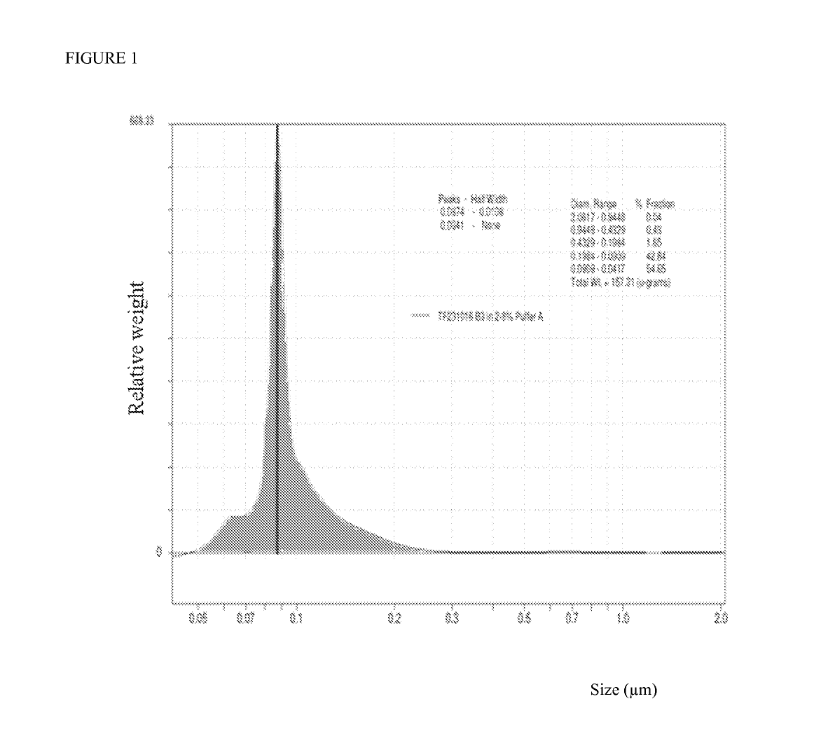

Having determined that correlations may be observed between the disc centrifugation-derived size distribution and the synthetic particle concentration, the inventors tested whether similar correlations may be observed using samples comprising virus particles. The virus used was influenza virus H3N2 (A/Uruguay/716/2007 X-175C) strain, which is non-aggregated (see FIG. 1). The original virus starting material was diluted in 50 mM sodium phosphate, pH 7.5, to 80%, 60%, 40% and 20% (v/v) of the starting material. Undiluted virus material was also used. The linearity of the maximum weight and the integrated weight of the size distribution of virus particles in the range of 40 nm to 2 μm was tested using various concentrations of virus particles. The original virus preparation comprising 1.37¹² protected copies/ml, as determined by qRT-PCR was tested. Also, original virus preparation was diluted in 50 mM NaPO₄ to provide 80%, 60%, 40%, 20% (v/v) dilutions compared to the original material. Undiluted virus preparation was also tested.

Results

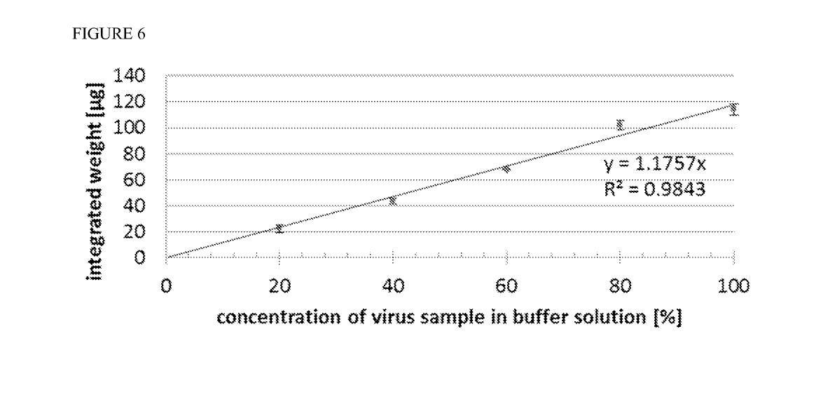

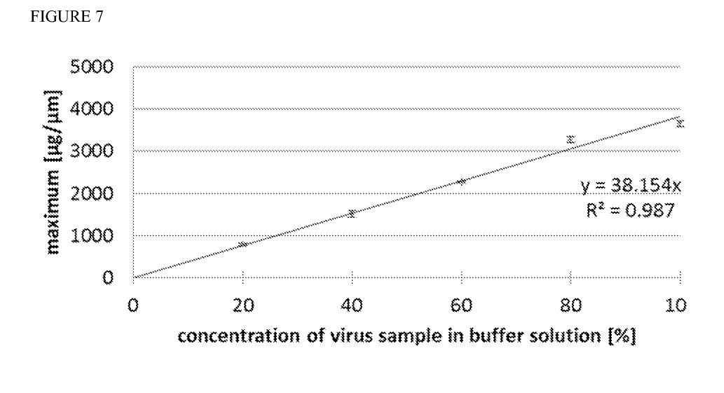

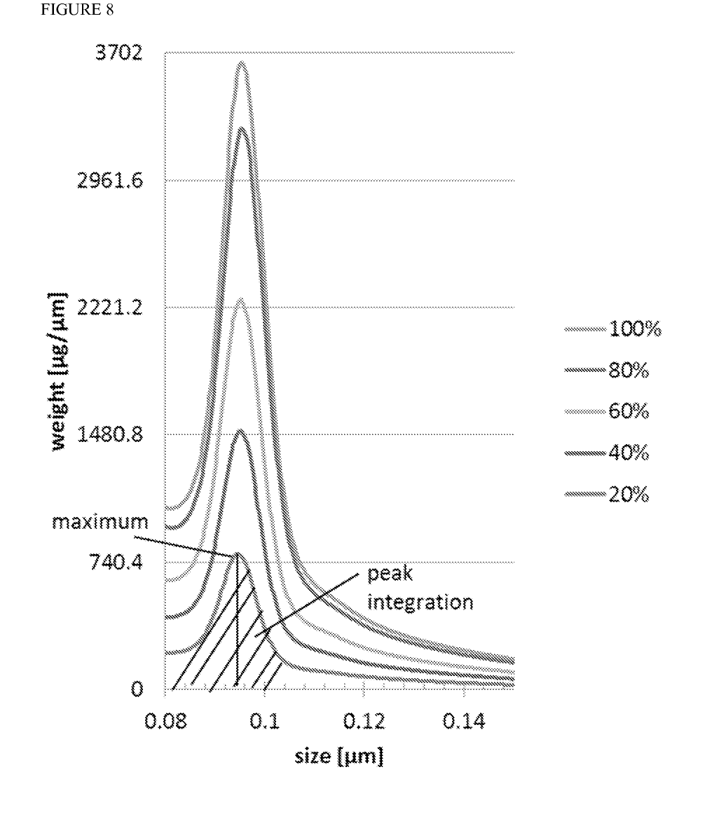

Results are shown in FIGS. 6-8. High correlation coefficients were observed for the integrated weight and for the maximum weight of the size distribution (0.9843 and 0.987 respectively) with the concentration of injected particles. This indicates a surprising linear relationship between the concentration of (non-aggregated) virus particles and size distribution characteristics. To evaluate reproducibility, all measurements were performed in triplicates, with a maximum standard deviation of 13%. With an estimated particle density of 1.16 g/cm³, a hydrodynamic diameter of about 95 nm was observed, which complies with the reference diameter between 80 and 120 nm [ref 45] and indicates singular particles. Aggregates in the measuring buffer were not observed.

Therefore, the maximum weight and the integrated weight of the size distribution correlate with the concentration of non-aggregated virus particles in the sample.

Influenza Samples Obtained by Various Downstream Processing Steps

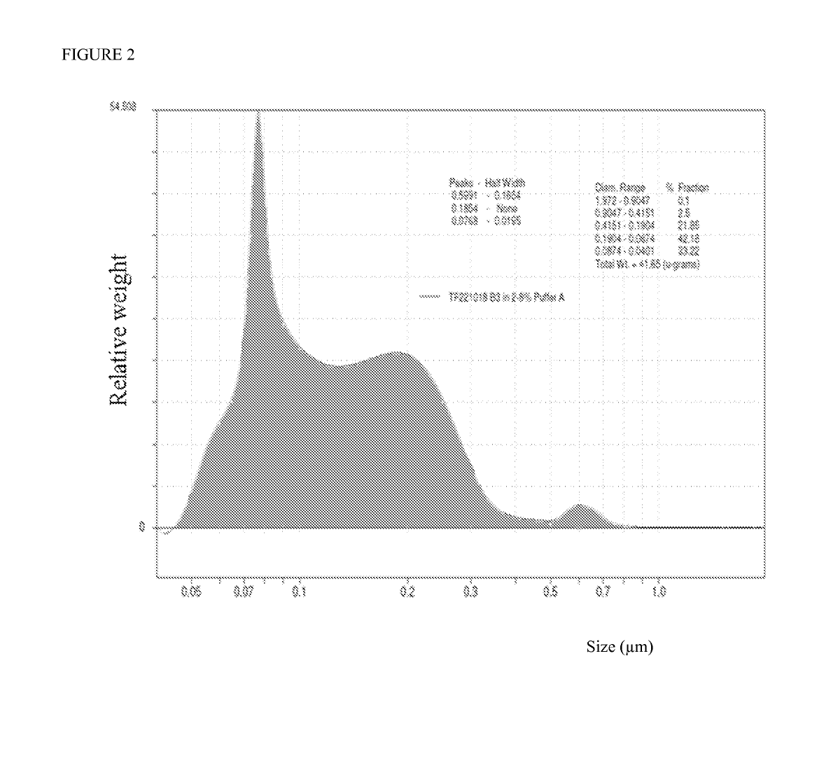

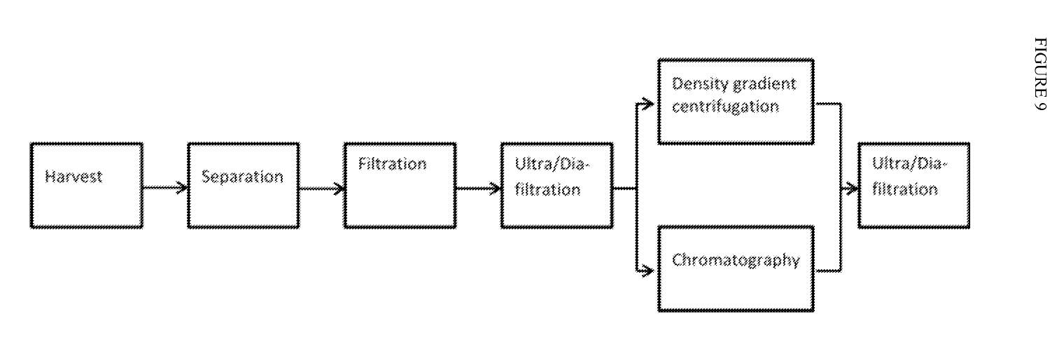

The inventors then investigated whether the correlations observed using a PVC standard and a non-aggregated single virus would translate to samples virus preparations that have undergone an additional processing step, containing different amounts of virus particles and different buffer conditions, including media, low salt and physiological buffer. The inventors tested different influenza preparations from three different strains (H3N2, H1N1 and a B-type) which were grown in MDCK cells. A summary of the downstream processing steps is provided in FIG. 9. H3N2 is a non-aggregating strain (FIG. 1), and H1N1 is an aggregating strain (see FIG. 2). The virus preparations were obtained by harvest, ultra- and diafiltration, chromatographical purification or density gradient purification, and were measured using disc centrifugation.

As an external measure for the quantification of virus particles, a reverse transcriptase qRT-PCR was used to determine the number of protected copies. As part of the sample pre-treatment, a RNase A/Tl mix (2 mg/ml of RNase A and 5000 U/ml of RNase Tl) (diluted 1:11 and incubated for 30 min at room temperature) was used to eliminate the influence of broken virus particles and free RNA. After dilution with CDM to a desired range for quantification, virus RNA was extracted using a QIAsymphony with the QIAsymphony Virus/Bacteria Midi Kit. 54 of extracted RNA was then added to 20 μL of the master mixture containing 0.40 μM of forward (nucleotide sequence: 5′-CAGGCCCCCTCAAAGC-3′ (SEQ ID NO: 1) for Influenza A, 5′-GTTTGGAGACACAATTGCCTACC-3′ (SEQ ID NO: 2) for Influenza B) and reverse primer (nucleotide sequence: 5′-GCGTCTACGCTGCAGTCC-3′ (SEQ ID NO: 3) for Influenza A, 5′-GCAGATAGAGGCACCAATTAGTG-3′ (SEQ ID NO: 4) for Influenza B) of a matrix gene conserved region, accordant TaqMan®-Probe 0.2 μM (nucleotide sequence: 5′-6FAM-AGGTGACAGGATTGGTCTTGTCTTTAGCC-BBQ-3′ (SEQ ID NO: 5) for Influenza A, 5′-6FAM-AGATGGAGAAGGCAAAGCAGAACTAGC-BBQ-3′ (SEQ ID NO: 6) for Influenza B), 12.5 μL reaction mix with a final concentration of 1×, 0.5 μL of SuperScript III RT/Platinum Taq Mix and 6.35 μL PCR grade water. The PCR reaction included an initial step for 15 minutes at 50° C. to allow reverse transcription of the single stranded RNA to double stranded cDNA and an initial Taq activation for 2 minutes at 95° C. Then, 45 cycles of 15 seconds at 94° C. and 45 seconds at 60° C. were performed. Fluorescence detection was carried out after each 60° C. step. For analysis, each reaction was characterized by its cycle threshold value. This identifies the cycle at which fluorescence crosses the predefined cycle threshold, which was 20 fluorescence units. Higher cycle threshold values could be correlated to lower logarithmic amount of initial standard invitrotranscipt, which was used for standard curve preparation. All reactions were quantified in relation to in vitro transcripts of known concentration.

The different virus samples contained different amounts of virus particles, as indicated by the number of protected copies per ml, ranging from 9.5E10⁹ to 2.9E10¹².

Quantification of hemagglutinin was performed using the single radial immuno-diffusion assay, as previously described [ref 42]. A gel, containing 1% agarose in Dulbecco's PBS and the specific antibodies, provided by the National Institute for Biological Standardization and Control (NIBSC, London), with 4 mm holes, was prepared. To prevent aggregation, each sample was treated with a 1% ((w/v)) zwittergent 3-14 solution. Samples were diluted to 75%, 50% and 25% (v/v) in relation to the original material using Dulbecco's PBS. Undiluted starting material was also used. 20 μL of sample was introduced into one cutting of the gel. After 18 hours of incubation, the gel was stained for 10 min in coomassie-blue-solution (1.5 g Serva Blue R in 500 mL destain solution) and destained for at least 30 min in destain solution (containg 11.8% ((v/v)) acetic acid and 30% ((v/v)) methanol in water). Next, the area of the rings with the antigen-antibody-precipitates were determined by measuring the diameter of the edges in two orthogonal directions. Analysis was performed using the slope ratio model, where the concentration of hemagglutinin can be quantified by the relation of the slopes of the sample and the reference standard of the diameter of the rings over the dilution. The quantification limit allowed only a hemagglutinin quantification of sufficiently high concentrated influenza samples.

Results for Non-Aggregating Influenza Strain (H3N2)

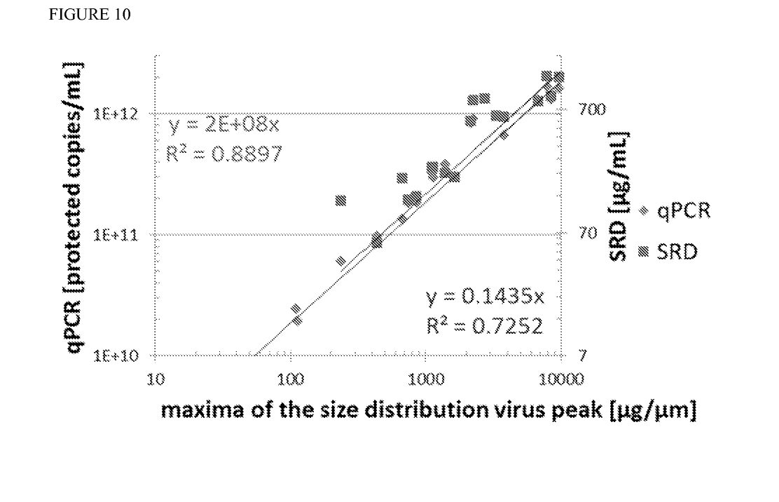

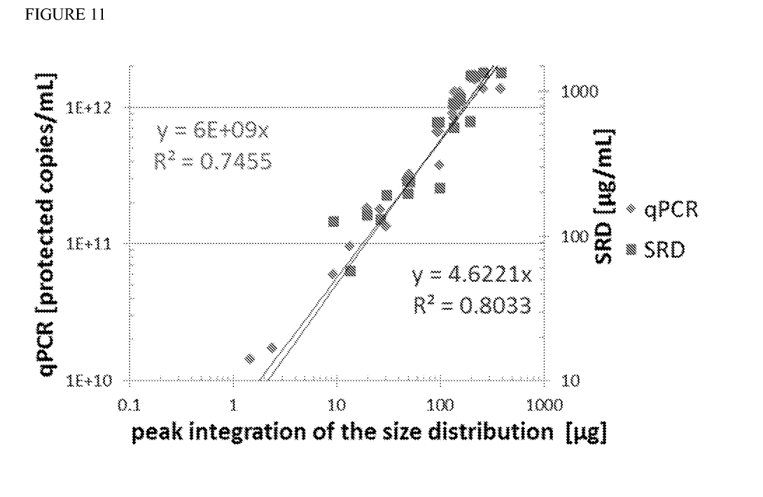

FIGS. 10-11 show the maximum weight and the integrated weight of the size distribution over the virus/hemagglutinin concentration for the H3N2-strain. No aggregates in the size distribution were observed (see FIG. 1), and a linear correlation between the amount of virus particles injected into the disc and the maximum of the size distribution was observed. High correlation coefficients of 0.8897 (qRT-PCR) and 0.7252 (SRD) were observed between the amount of injected virus particles and the maximum weight of the size distribution. High correlation coefficients of 0.7455 (qRT-PCR) and 0.8033 (SRD) were also observed between the amount of injected virus particles and the peak integration. These indicate a linear relationship between the amount of injected virus particles and size distribution characteristic factors. Note that for samples containing low concentrations of influenza particles, hemagglutinin concentration was not high enough to reach the quantification limit of the SRD assay, so that the x-range were smaller, resulting in lower R2 values. Furthermore, it has to be considered, that the SRD and qRT-PCR values are measured with a typical standard deviation of less than 10% respectively 20%, thus lowering the upper limit of the correlation coefficient related to the correlation to known values.

Therefore, the inventors observed a linear relationship between the amount of injected non-aggregated virus particles and size distribution characteristics (maximum weight of the size distribution and integrated weight of the size distribution).

Results for Aggregating Influenza Strain (H1N1)

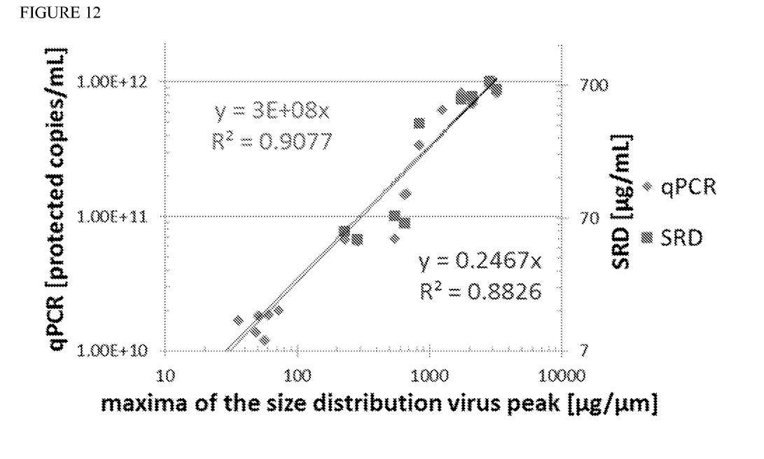

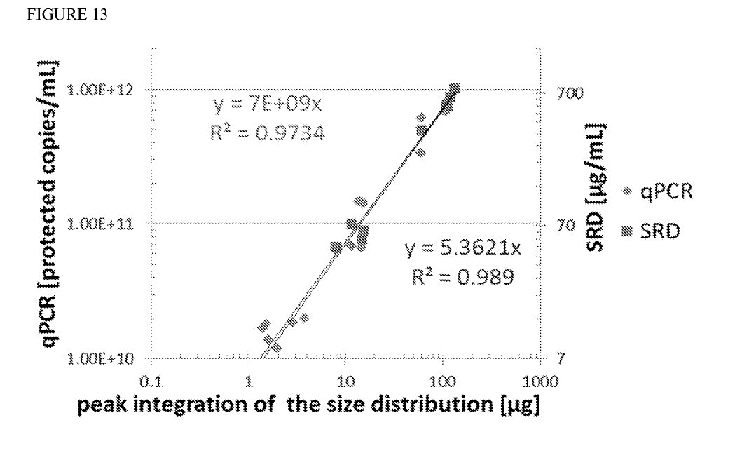

Samples were obtained by different production techniques, including harvest, ultra/diafiltration, chromatographical purification and density gradient purification (FIG. 9). These samples therefore contained different buffers and different ratios between aggregated and non-aggregated virus particles. As for the H3N2 strain, a linear correlation was observed between the amount of injected particles and the maximum weight of the size distribution, with a correlation coefficient of 0.9077 (qRT-PCR) and 0.8826 (SRD) (see FIGS. 12 and 13). A linear correlation was observed between the amount of injected particles and integrated weight of the virus peak 0.9734 (qRT-PCR) and 0.989 (SRD).

As shown below, the choice of buffer used for the density gradient preparation influences the conformation of the particles, and so it is important to maintain the same amount of aggregates relative to the amount of singular particles. Ideally all particles are non-aggregated and therefore the use of one measuring buffer for all samples can ensure equal buffer refractive index and influences in aggregation. The high correlation coefficients indicate that the size distributions of the different influenza-containing samples (with different buffer conditions used during processing) are mainly a function of the density gradient buffer in the disc centrifuge, so long as no irreversible aggregating conditions were used during pretreatment. The inventors found that under conditions that induce aggregation, the linear relationship between maximum weight of the size distribution and the amount of injected aggregated virus was lost. Despite this, the inventors chose to use an integration interval which is large enough to include aggregated particles. The inventors surprisingly found that size distribution still correlates with the amount of injected virus particles in the sample, but only for the integrated weight of the size distribution (FIG. 13). This provides a surprising new way of quantifying samples comprising virus particles which contain a mixture of non-aggregated and aggregated virus particles, which has not been easily achieved in the prior art.

The inventors also observed that a unimodal size distribution can provide a positive control for the nonexistence of particle aggregation. This may also be used to verify other quantitative assay data, for which the area:volume ratio may influence the measurement, e.g. ELISAs.

Results for Aggregating Influenza B Strain (B/Brisbane/60/2008)

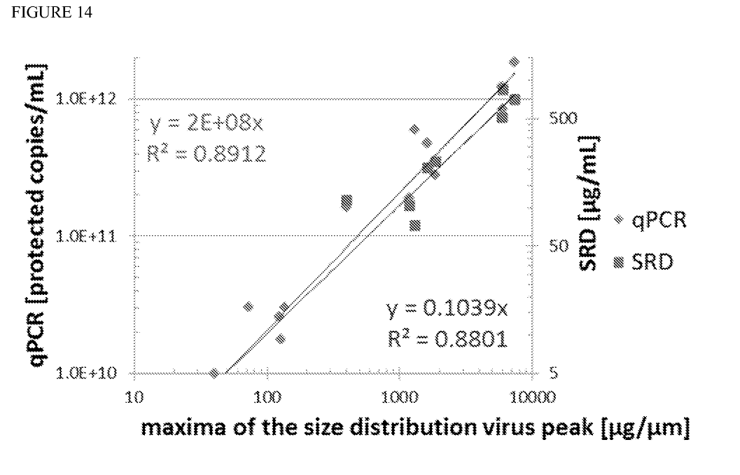

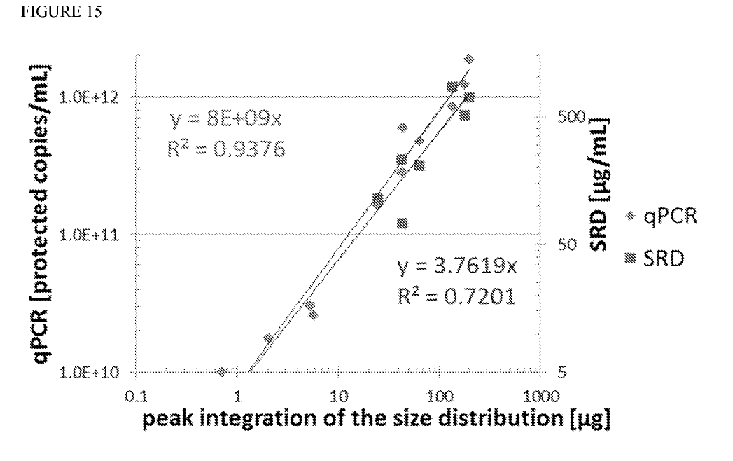

This influenza B strain showed no aggregation when using 50 mM sodium phosphate pH 7.5 buffer for gradient preparation, when using harvest, separated harvest, ultra/diafiltrated and density gradient or chromatographical purified material. The different virus-containing samples contained different amounts of virus particles, as indicated by the number of protected copies per ml, ranging from 1E10 to 2E12, as measured by qRT-PCR. High correlation coefficients (0.8912 with qRT-PCR and 0.8801 with SRD) were observed between the maximum weight of the size distribution and the injected amount of protected particles per mL (see FIG. 14). High correlation coefficients were also observed between the integrated size distribution and the injected particle concentration, with values of 0.9376 (qRT-PCR)/0.7201 (SRD) (see FIG. 15). These strong correlations indicate a strong linear relationship between these size distribution parameters and the concentration of injected virus.

Results for all Tested Influenza Strains

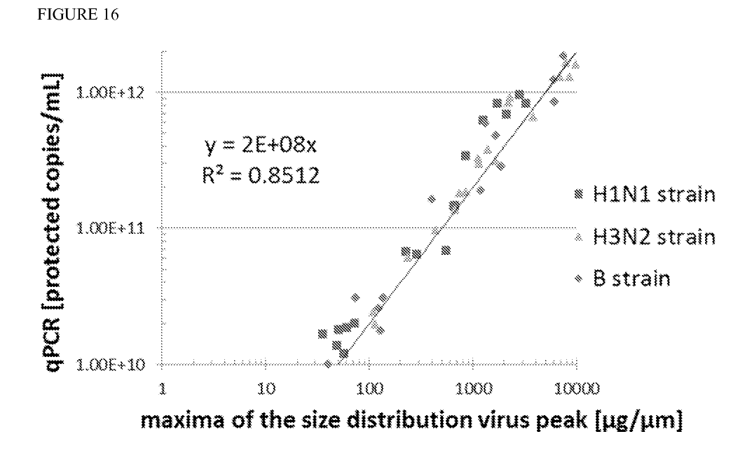

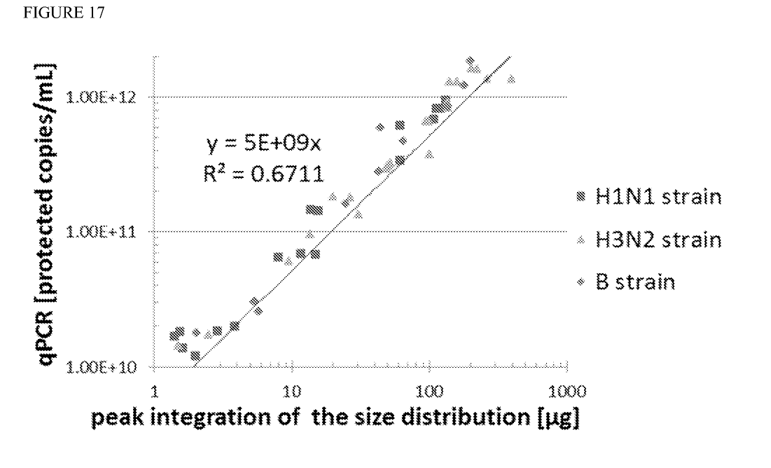

All tested influenza strains belong to the same species and have only slight differences in morphology and structure, as demonstrated in FIGS. 16 and 17. Although the H1N1 strain showed aggregation in the 50 mM sodium phosphate pH 7.5 buffer, the addition of 120 mM NaCl reduced aggregation, allowing a correlation between the maximum weight of the size distribution and the injected particle concentration. The addition of 120 mM NaCl did not affect the size distributions of the other tested strains, and so other parameters which influence the measurements, such as refractory index, remain constant. All tested influenza strains showed similar slopes of the linear relationship between the concentrations of protected copies per ml and the maximum weight of the size distribution, and also for the integrated weight.

Where the conformational state of the virus particles is mostly non-aggregated, (which can be easily determined from the size distribution), different strains of influenza particles may be quantified without strain-specific calibration. However, due to slight strain-specific differences, correlation coefficients may be higher if different strains are assessed separately. Furthermore, different strains may have different ratios of non-aggregated to aggregated particles, or different size ranges, leading to peaks with different breadths, and therefore amplitudes. So long as the same resolution and ratio of non-aggregated to aggregated particles is ensured, e.g. where the same gradient solutions is used for all measurements, quantification based on the maximum weight of the size distribution can suffice. For some particle quantification applications, such as mass balancing in the production of one strain, the relative quantity may be used, rather than the absolute quantity.

The Influence of Buffer Choice on Aggregate Formation

The inventors tested whether the choice of buffer used in the separating fluid (e.g. a density gradient) can influence the conformation of the measured particles.

Results

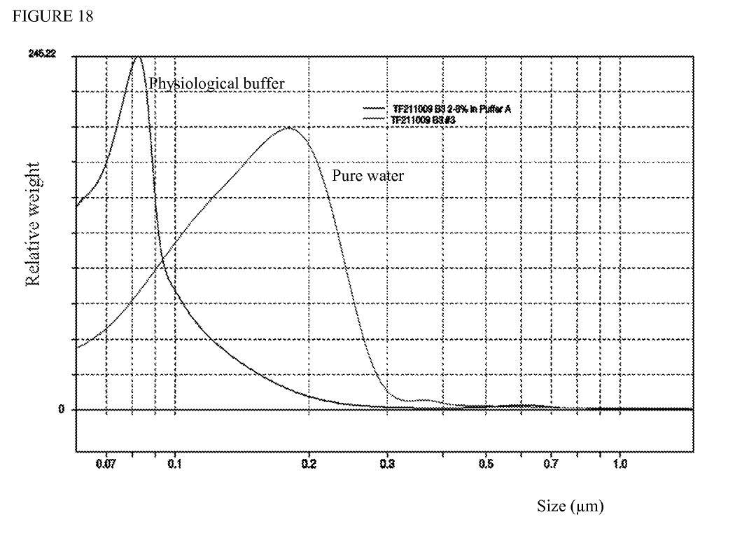

In the present examples, the physiological buffer (50 mM NaPO₄, pH 7.5) induced slight aggregation of an H1N1 strain, (aggregates as well as singular, non-aggregated particles were observed, FIG. 18).

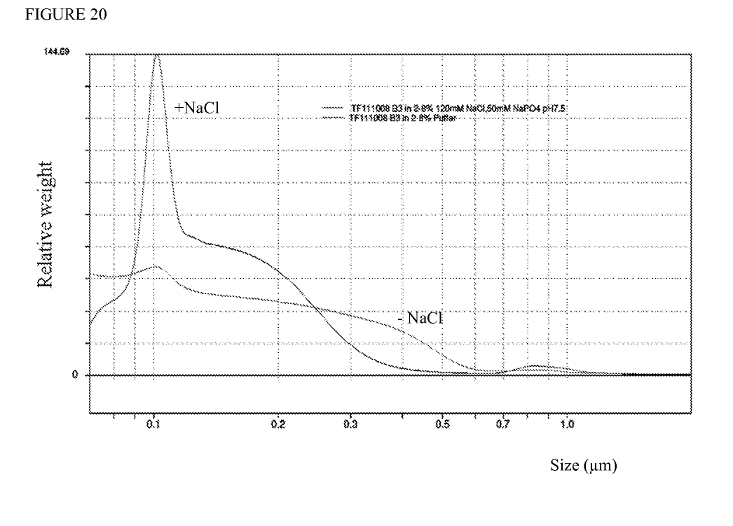

The addition of NaCl to a final concentration of 120 mM led to a decrease of aggregates (FIG. 20) and an increase in non-aggregated particles, as indicated by the increased maximum of the size distribution at the virus size. This reaffirms the inventors' observation that the buffer used for density gradient preparation has a large influence on size distribution measurements.

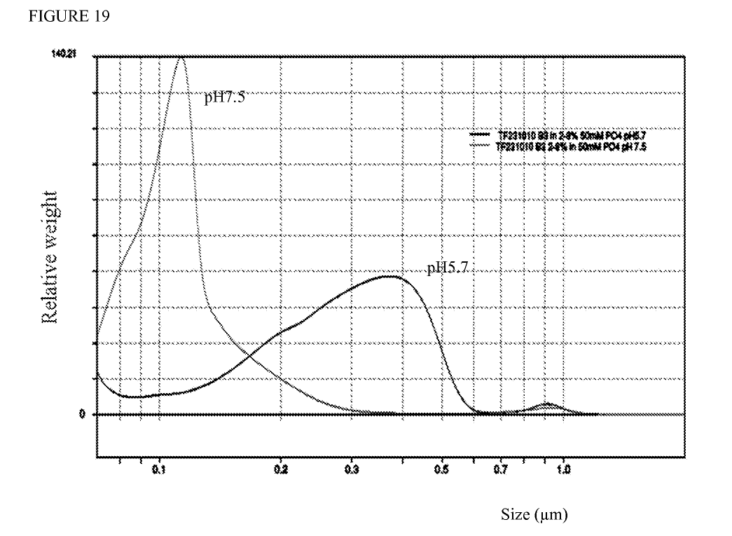

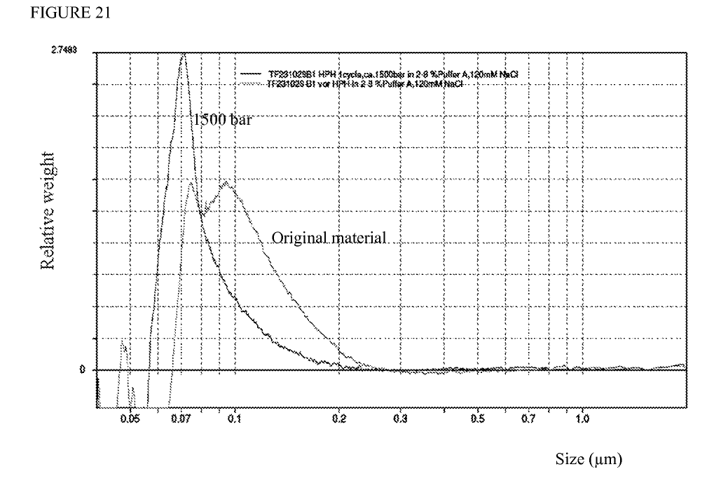

Preparing the density gradient in pure water (FIG. 18) or with a low pH (FIG. 19) induced strong virus aggregation, as described in reference 43. Conversely, homogenization using a high pressure homogenizer, as described in reference 1, led to a reduction in aggregation and an increase in the number of non-aggregated particles, as indicated by higher maximum weight of the size distribution peak and a higher TCID₅₀/ml (FIG. 21).

For aggregated particles, disaggregating conditions/processes such as sonication, high pressure homogenization (see reference 1), or the addition of salts could be used to help minimise aggregation. It may be possible to quantify aggregating viruses by the maximum weight of the size distribution for a dominant virus peak. This could be beneficial because non-aggregated viruses (FIGS. 10-11) showed a higher correlation coefficient than aggregated viruses, with size distribution characteristics which could provide further increased specificity. Using an integrated weight distribution is more susceptible to the inclusion of cellular particles or aggregates of virus particles involving cellular components.

The Benefit of Disc Centrifugation Over Methods in the Prior Art

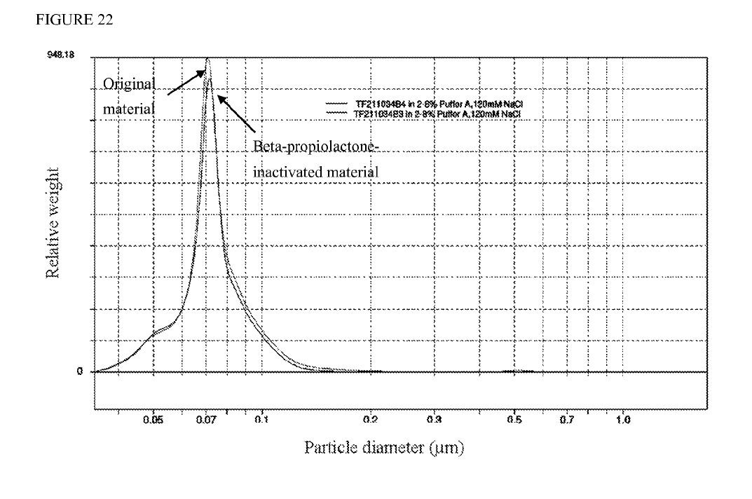

The inventors tested how the disc centrifuge method of the present invention compares to virus quantification methods in the prior art, including the current gold standard for virus quantification, qRT-PCR. Beta-propiolactone-inactivated influenza virus samples were measured using the disc centrifuge method of the present invention, TEM, and the current gold standard, qRT-PCR. The amount of virus particles measured using qRT-PCR and TEM is shown in Table 1, and the amount of virus particles measured using the disc centrifugation method of the present invention is shown in FIG. 22.

TABLE 1
Quantification of beta-propiolactone-inactivated influenza virus samples
using qRT-PCR and TEM

| | qRT-PCR | |
|---|---|---|
| Material | [protected copies/ml, Std dev] | TEM [virus particles/ml] |
| Original | 1.612¹², 16% | 1.6¹¹ |
| Inactivated | 9.713¹¹, 5% | 1.3¹¹ |
| % inactivated | 57 | 81 |

Beta-propiolactone-inactivation damages the RNA of influenza virus. Quantification by qRT-PCR provides a value that is 43% lower than the number of particles actually present in the original sample. Although time consuming, TEM was more accurate than qRT-PCR, and provided a virus count that is 81% of the original particle number. Surprisingly however, the disc centrifuge-based method of the present invention proved to be far easier and more accurate than conventional techniques in the prior art, and detected 93% of the original particle number, when using the maximum weight of the size distribution, and 92% when using the integrated weight of the size distribution.

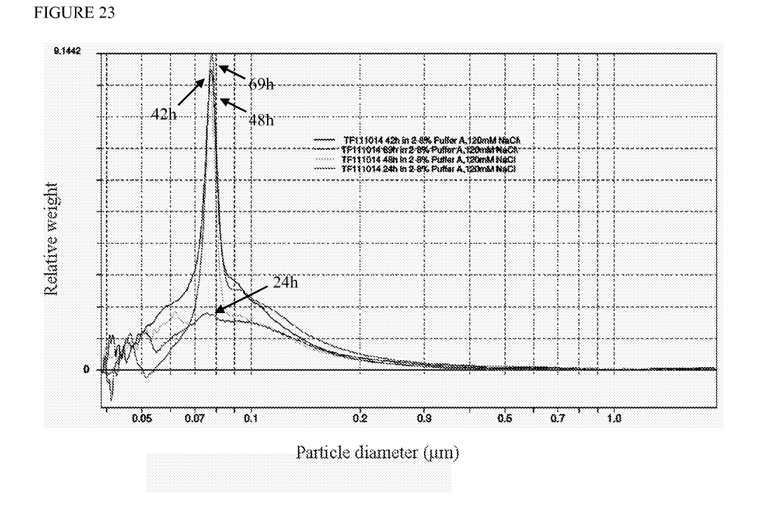

Thus, the present invention provides a rapid and accurate method for quantifying viral content, which is better than methods available in the prior art. The present invention is also useful for monitoring whether whole virus particles were destroyed, as indicated by, e.g. a relative increase in the proportion of particle sizes that are smaller than the original virus starting material. The present invention is also useful for monitoring levels of a virus in a bioreactor as shown in FIG. 23. FIG. 23 also demonstrates that the present invention is particularly useful for quantifying high titer fermentations. Based on FIG. 23, the detection limit of the method, using the instrumentation detailed herein, was evaluated to be approximately 5E09 protected virus copies/ml, as measured by qRT-PCR. The detection limit may vary according to the type of particles in the sample and the instrumentation used to determine the particle size distribution.

### Conclusion

The present invention provides a fast method for quantifying non-aggregated and/or aggregated virus particles in a sample that is based on a physical, particle measuring method. The method of the invention makes it possible to quantify samples comprising virus particles, e.g. influenza particles, in less than 15 minutes. The inventors surprisingly found that disc centrifugation, in combination with a detector, such as a light absorption detector, can be used to quantify virus particles, such as influenza virus particles. Under the experimental conditions used herein, the H3N2 strain and B strain of influenza showed a unimodal size distribution which is indicative of a non-aggregated virus. A high correlation was observed between the amount of influenza particles, (as determined by qRT-PCR), the amount of hemagglutinin (as measured by SRD), and the maximum weight of the size distribution and also the integrated weight of the size distribution.

Under the experimental conditions used herein, the H1N1 strain of influenza showed aggregation, when using 50 mM sodium phosphate buffer, pH 7.5. A strong correlation was observed between the integrated weight of the size distribution (comprising non-aggregated and aggregated particles) and the amount of injected virus particles. The inventors also found that aggregation could be decreased by the addition of 120 mM NaCl, which resulted in a strong correlation between the integrated weight of the size distribution, and also the maximum weight of the size distribution, with the amount of injected virus particles/hemagglutinin. The method of the invention is suitable for use in virus production e.g. for monitoring the quantity of virus particles.

Furthermore, the method of the present invention showed linearity between the amount of injected particles and size distribution characteristic factors, which minimizes the errors often encountered when using existing virus quantification methods, such as ELISA or virus titration, where logarithmic dilution steps are required for the titer estimation, thus resulting in a higher error due to logarithmic scaling. Similarly, serial dilution steps that are often required for quantification methods in the prior art can enhance measurement error due to the errors that are introduced with each dilution step.

Determination of Particle Density and Particle Size by Disc Centrifugation

The inventors also tested whether disc centrifugation may be used to determine particle density and also particle size e.g. virus particles. This would eliminate the need for externally measured (e.g. by any of the virus quantification methods in the prior art), or literature-derived particle densities. This method is useful for determining e.g. particle density and/or size. Particles may be biological e.g. virus particles, or non-biological.

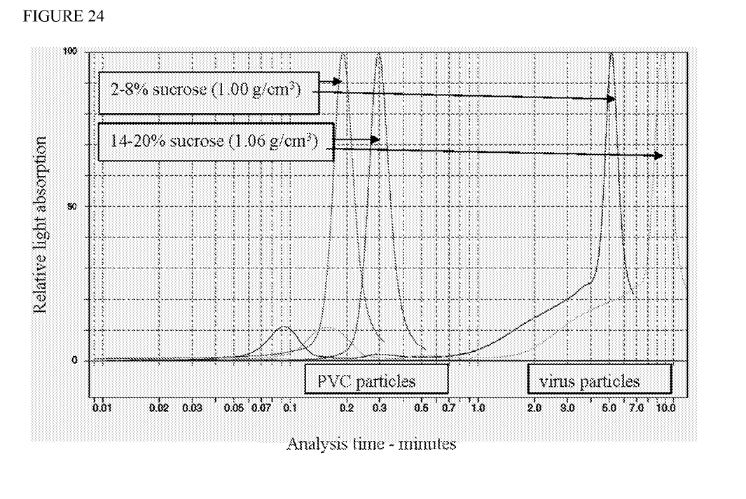

These experiments were based on several principles. The time between particle injection and particle detection may be used to determine the settling velocity of the particles. The settling velocity may be correlated to particle size. In particular, particles in a fluid with higher viscosity and density have a longer settling time until detection by the detector (FIG. 24). The rheological properties of the separating fluid (density, viscosity etc.) may be calculated using a standard which has a known density and size (hydrodynamic diameter) e.g. PVC standard particles. Therefore, only a small set of parameters are required to measure particle density and particle size by disc centrifugation, compared to other size measuring techniques in the prior art e.g. dynamic light scattering. The critical parameter is the buoyant density.

The inventors tested whether altered settling velocities for particles in fluids which have different viscosities and/or densities, makes it possible to perform a regression analysis for the calculation of particle densities.

Operation of the Disc Centrifuge

For the determination of particle density and particle size by disc centrifugation, a 2-8% (w/v) and a 14-20% (w/v) sucrose gradient in 50 mM NaPO₄, pH 7.5 was prepared by mixing the upper concentrated solution with the lower concentrated solution to provide a 9 solutions (with 2%, 2.75%, 3.5% . . . 8% sucrose and 14%, 14.75%, . . . , 20% sucrose respectively). For the non-biological material, additional measurements in 31-37% (w/v) and 51-57% (w/v) sucrose gradients in 50 mM NaPO₄, pH 7.5 were performed. Rotation of the disc was set to 24000 RPM for biological particles, and 10000 rpm for non-biological materials. After reaching the set RPM, 1.6 mL of each sucrose solution, beginning with the highest concentration and ending with lowest concentration solution, was injected into the disc. 0.5 ml of dodecane was then injected to prevent the evaporation of water. After 20-30 min of equilibration time, the measurement was started. 226 nm and 263 nm standard PVC particle solutions containing 0.05% PVC were used for calibration before characterisation. 100 μl standard particle solution was measured before and after sample measurement for subsequent data analysis. Measurements using the second sucrose buffer gradient were then performed. A Hamilton syringe was used for all sample injections.

Particle Solutions

The following virus particle solutions were used: one Influenza B (strain B/Brisbane/60/2008) and two Influenza A (H3N2) strains (A/H3N2, strain A/Uruguay/716/2007 X-175C and strain A/Victoria/210/2009 X-187), grown in MDCK cells (33016 PF) in CDM/PF medium; Tobacco mosaic virus (TMV), grown in tobacco plants; Tomato mosaic virus (ToMV), grown in tobacco plants; Rabies (Flury LEP) virus, grown in chicken embryo fibroblasts. The following PVC standards were used: polyvinyl chloride particle solution, 377 nm and polyvinyl chloride particle solution, 226 nm. Silicon oxide was also used.

Results

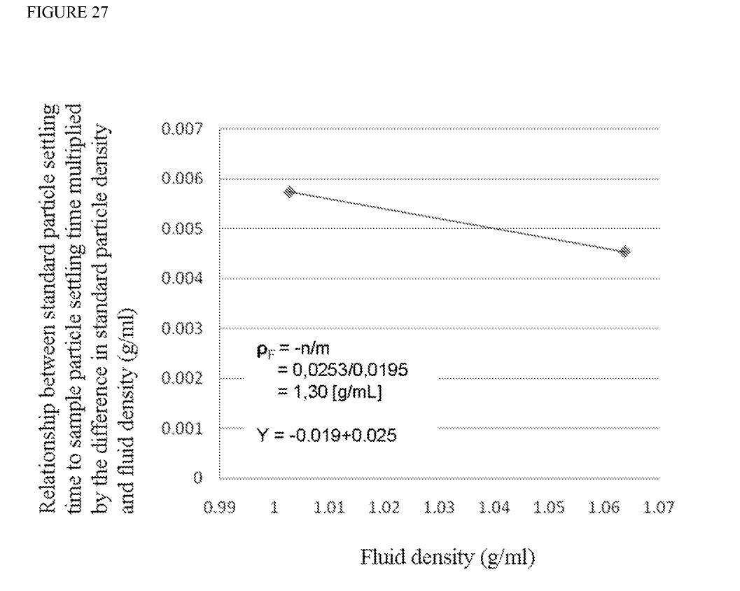

The inventors derived a model which allows the estimation of the buoyant density, and therefore the absolute correct size (hydrodynamic diameter), of particles in a sample. The model is based on (at least) a measurement of the sample in (at least) two different fluids which have different rheological properties, followed by a regression analysis. Particles in a separating fluid with a higher viscosity and density were found to exhibit a longer settling time until detection (see FIG. 27) and vice versa.

The applicability of this model for the size determination of PVC-particles and virus particles is demonstrated by comparison of the estimated buoyant densities and size with reference values in the literature (Table 2).

Modelling the Particle Density and Particle Size Measuring Process with Constant Viscosity

Sedimentation in steady state can be described by balancing the drag force according to Stokes' law:

$$\begin{matrix}{F}_{s} = 3 π D η v \\ = 3 π D η \frac{\partial R}{\partial t}\end{matrix} \qquad ( 1 )$$

where:

Fₛ=drag force

D=diameter

q=dynamic viscosity

R=radius

t=settling time

v=settling velocity

and the centrifugal force:

$${F}_{G} = π / 6 ) {D}_{3}^{↑} ( {ρ}_{↓} P - {ρ}_{↓} F ) {ω}^{↑} 2 r \\ {F}_{c} = \frac{π}{6} {D}^{3} ( {ρ}_{P} - {ρ}_{F} ) {ω}^{2} R \qquad ( 2 )$$

where

F_{c}=centrifugal force

Pp=particle density

ρ_{F}=fluid density

ω=angular acceleration

R=radius

leading to:

$$\begin{matrix}t = \frac{X}{v} \\ = \frac{18 X η}{{D}^{2} {ω}^{2} r ( {ρ}_{P} - {ρ}_{F} )}\end{matrix} \qquad ( 3 )$$

Which, after integration yields:

$$= \frac{\partial R}{R} = \frac{{D}^{2} {ω}^{2} ( {ρ}_{P} - {ρ}_{F} )}{18 η} \partial t = {\int }_{{R}_{0}}^{{R}_{D}} \frac{\partial R}{\partial } = \frac{{D}^{2} {ω}^{2} ( {ρ}_{P} - {ρ}_{F} )}{18 η} {\int }_{{t}_{0} = 0}^{{t}_{D} = t} \partial t = ln \frac{{R}_{D}}{{R}_{0}} = \frac{{D}^{2} {ω}^{2} ( {ρ}_{P} - {ρ}_{F} )}{18 η} \\ t = \frac{18 ηln \frac{{R}_{D}}{{R}_{0}}}{{D}^{2} {ω}^{2} ( {ρ}_{P} - {ρ}_{F} )} \qquad ( 4 )$$

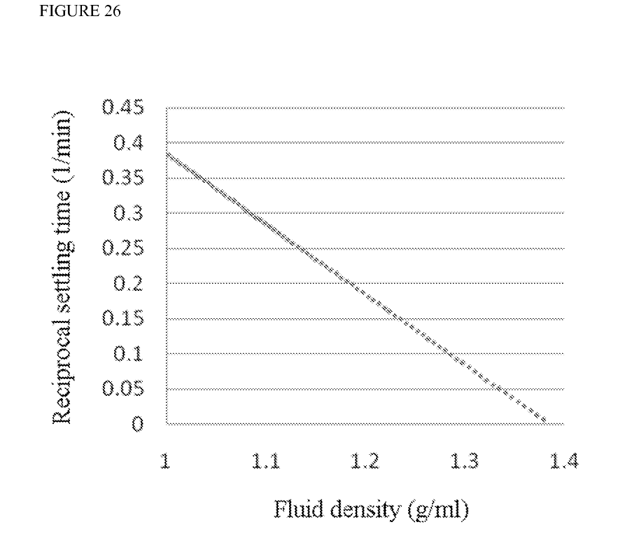

As an example, for particles with a density of 1.385 g/ml, a constant settling distance (point of detection R_{D} and initial settling point R₀), viscosity, diameter and angular acceleration, the settling time t over the fluid density is shown in FIG. 26. At a particle density equal to the fluid density, the settling time approaches zero due to the decrease in the centrifugal force. Thus, the settling time converges to infinity.

Reciprocal transformation of the settling time over fluid density leads to a linear relationship:

$$\begin{matrix}\frac{1}{t} = \frac{{D}^{2} {ω}^{2} r ( {ρ}_{P} - {ρ}_{F} )}{18 X η} \\ = - {m}_{0} {ρ}_{F} + {n}_{0} , {n}_{0} \\ = {m}_{0} {ρ}_{P} , {m}_{0} \\ = \frac{{D}^{2} {ω}^{2} r}{18 X η}\end{matrix} \qquad ( 5 )$$

(see FIG. 26). Thus, by plotting 1/t over the density of the fluid (e.g. by weighing 1 ml of separating fluid) and a regression analysis, the particle density may be estimated for constant viscosity by dividing the absolute term of the regression by the negative slope:

$${ρ}_{p} = \frac{{n}_{0}}{{m}_{0}} \qquad ( 6 )$$

Therefore, measuring the same sample in two different separating fluids which have different (but known) densities and constant viscosity, at the same speed, and the same amount of fluid in the disc will lead to a regression where buoyant density can be estimated. Constant viscosity for two fluids which have different densities is a strong assumption. The next step provides generalisations in the model to allow sample measurement in two fluids which have different rheological properties, especially viscosity.

Modelling the Particle Density and Particle Size Measuring Process with Variable Viscosity Avoiding External Measurement of Viscosity

With variable, but known viscosity, the model in equation (4) can be derived to:

$$\frac{η}{t} = \frac{{D}^{2} {ω}^{2} r ( {ρ}_{P} - {ρ}_{F} )}{18 X} \qquad ( 7 )$$

Here, with known viscosities, a plot of η/t versus ρ_{F} would lead, in the same way as above, to the calculation of unknown particle density. However, the fluid viscosity is highly dependent on a number of parameters e.g. temperature and chemical composition of the fluid. Accordingly, working with data in the literature may result in high errors, assuming such data is available at all. Therefore, an estimate for the separation fluid viscosity is required. Since a standard e.g. PVC particles, are required for calibration, the viscosity for the fluid can be derived by:

$$η = \frac{{D}_{Std}^{2} {ω}^{2} r ( {ρ}_{P , Std} - {ρ}_{F} ) {t}_{Std}}{18 X} \qquad ( 8 )$$

Where

Pp,Std=calibration standard density

D_{Std}=calibration standard diameter

t_{std}=settling time of calibration standard

Inserting (8) into (7) leads to:

$$\begin{matrix}\frac{( {ρ}_{P , Std} - {ρ}_{F} ) {t}_{Std}}{t} = \frac{{D}^{2}}{{D}_{Std}^{2}} ( {ρ}_{P} - {ρ}_{F} ) \\ = - m {ρ}_{F} + n , n \\ = m {ρ}_{p}\end{matrix} \qquad ( 9 )$$

Thus, by plotting the difference between the standard particle density and fluid density multiplied by the quotient of settling time of standard particles divided by sample particle settling time over the fluid density, the particle density can be estimated by dividing the slope of the linear regression by the y-intercept:

$${ρ}_{p} = \frac{n}{m} \qquad ( 10 )$$

Only the calibration standard density has to be known, the calibration standard size has to be the same for all tested fluid densities. Furthermore, equation (8) also allows the estimation of particle hydrodynamic diameter in relation to the calibration standard size by:

D=\sqrt{m*D²} (11)

Conditions necessary for applying this model are:
Constant settling distance from starting point of settling to detection of particles, which is, in fact, the application of the same fluid volume injected into the disc for the applied gradient fluids.
At least two separating fluids with different rheological properties (density, viscosity etc.).
Density of separating fluids must be known.
Application of standard particles with known density.
Constant rotations per minute for all measurements.
No changes in diameter between both fluids (for example no aggregation of particles in buffer 1 and no aggregation in buffer 2). This includes streaming, which can occur if a sample with a higher total density than the lowest density of the density gradient in the disc is brought into the disc. The sample itself, or parts of the sample fluid, can behave as a particle.

All these necessary conditions are easy to fulfil except, perhaps, the last condition. Nevertheless, aggregation, or at least a shift in size, can be easily identified by size distribution analysis. Where aggregation has taken place, this can be identified by drastically shifted distribution sizes, or much broader peaks. Although working with “incorrect” densities, particles of same size should have same “incorrect” sizes in different fluids. Streaming effects or other size changing processes e.g. aggregation can otherwise occur.

Fluid densities are relatively easy to determine, and not as sensitive as the viscosity to temperature changes. However, temperature differences during the external density characterization and over the time period of the disc centrifugation are difficult to control and might affect the measurement. Alternatively, fluid densities can be determined by the introduction of a second calibration standard. For this method, the density and diameter has to be known for both calibration standards. Here, the standard particle density instead of the fluid density is altered without a change in centrifugation speed, fluid density, fluid viscosity and distance from settling starting point to detection to allow for a linear regression analysis:

$$\begin{matrix}\frac{1}{{tD}^{2}} = \frac{{ω}^{2} ( {ρ}_{P} - {ρ}_{F} )}{18 ln \frac{{R}_{D}}{{R}_{0}} η} \\ = - {m}_{1} {ρ}_{P} + {n}_{1} , {n}_{1} \\ = {m}_{1} {ρ}_{P}\end{matrix} \qquad ( 12 )$$

Again, plotting 1/tD2 over p_{P} leads by division of the negative y-intercept by the slope of the linear regression to the fluid density:

$${ρ}_{F} = \frac{{n}_{1}}{{m}_{1}}$$

Since fluid density could easily be measured and a second calibration standard was not available, this method was not further evaluated. Nevertheless, this method would completely avoid the need for externally measured parameters using two calibration standards.

Results

The measured particle densities of various virus and non-biological particles are shown in Table 2. The regression analysis for the estimation of the particle density of tomato mosaic virus is shown in FIG. 22, and the regression analysis for the estimation of the particle density of PVC particles is shown in FIG. 23. For all measured particles, the error of the estimated density compared to the “real” density, according to reference 45 was less than 5%, demonstrating an optimal size approximation by disc centrifugation.

TABLE 2
Densities and sizes of virus particles, PVC and silicon oxide,
compared to literature values. 1 = reference density for viruses from
“Springer Index of Viruses”, 2 = Stokes' diameter calculated from
equation 14, 3 = multimodal distribution, no single diameter available.

| | | | Stokes' diameter | |
|---|---|---|---|---|
| | | | determined | |
| | | | by disc | |
| | Density | | centrifugation | |
| | determined by | | based on | Reference |
| | regression | Reference | density | stokes' |
| | analysis | density | determination | diameter |
| Particles | (g/dm3) | (g/dm3) | (nm) | (nm) |
| ToMV | 1.30 | 1.325 | 37 | 34 2 |
| A/Uruguay/ | 1.19 | 1.20 | 75 | 80-120 |
| 716/2007 | | | | |
| X-175C | | | | |
| A/Victoria/ | 1.18 | 1.20 | 79 | 80-120 |
| 210/2009 | | | | |
| X-187 | | | | |
| B/Brisbane/ | 1.17 | 1.20 | 83 | 80-120 |
| 60/2008 | | | | |
| TMV | 1.28 | 1.325 | 40 | 34 2 |
| Rabies | 1.16 | 1.19-1.20 | 87 | 94 2 |
| PVC | 1.40 | 1.385 | 360 | 377 |
| Silicon | 2.30 | 2.0-2.1 | −3 | −3 |
| Oxide | | | | |

The present invention provides a strong approximation particle size by disc centrifugation. Rabies virus, TMV and ToMV are rod shaped and elongated particles, so the hydrodynamic diameter is not available in the literature. Nevertheless, according to reference 44, the hydrodynamic diameter of rod-shaped particles can be estimated by:

$$\begin{matrix}{d}_{Stokes} = {d}_{c} \sqrt{ln ( 2 β )} \\ = {d}_{c} \sqrt{ln ( 2 \frac{l}{{d}_{c}} )}\end{matrix} \qquad ( 14 )$$

where
dₛₜₒₖₑₛ=Stokes' diameter
d_{c}=cylindrical diameter=
l=length=
β=length/axis ratio

Accordingly, ToMV and TMV have lengths of 300 nm and diameters of 18 nm (see reference 45) and are therefore expected to have a hydrodynamic diameter of 34 nm. Rabies virus is reported to have a length of 160 nm and a diameter of 75 nm, and so is expected to have a hydrodynamic diameter of 94 nm. Since ToMV and TMV belong to the same virus family, similar densities and sizes are expected.

The difference between the estimated density and estimated size between the ToMV and TMV strains is less than 2%, with an error related to the real density of less than 5%, and an error related to the size of less than 20%. Similarly, the difference between estimated densities and literature values (according to reference 45, was less than 3%. Also, for rabies virus, the estimated density and real density had an error of less than 4%, leading to a similar size with an error related to the calculated real hydrodynamic diameter of less than 13%.

The evaluated non-biological particles were polyvinyl chloride and silicon (IV) oxide powder, for which an error between the estimated density and reference density was less than 2% (polyvinyl chloride) and 15% (silicon (IV) oxide). Since use of particle standards enables direct measurements of most relevant parameters by disc centrifugation (see reference 46), a reproducible and correct size distribution can be obtained. For the applied virus particles, the reproducibility of the measured size as indicated by the standard deviation was found to be less than 2% (measured in triplicate, data not shown). Furthermore, the greater the density differences between the fluids, the lesser the influence of the errors produced by a deviation in y-direction. Finally, the fluid density at the point of detection has to be lower than the target particle density to avoid trapping of particles within the density gradient.

Thus, disc centrifugation in combination with the use of particle standards, directly measures most of the parameters required to determine particle size and particle density (see also reference 46). A reproducible and correct size distribution can therefore be obtained using this method of the invention. The inventors found that this method is particularly useful for the analysis of virus particles, giving a standard deviation of measurements less than 2% (data not shown), indicating that the method is highly reproducible. Also, data obtained through this method complies with manufacturer information.

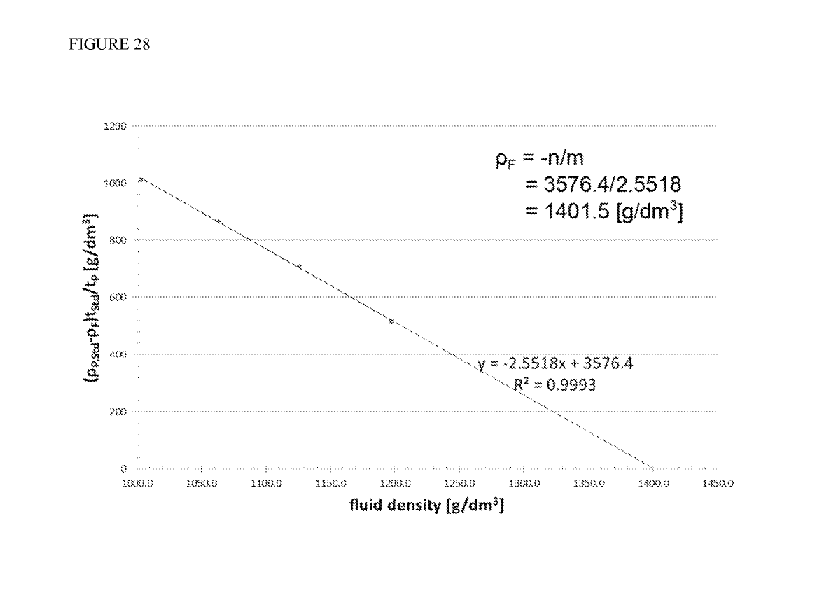

Although an error approximation was not performed, a number of factors could be used to optimize a density estimation using the disc centrifugation e.g. a larger difference between the densities of the fluids should decrease the influence of any errors produced by a deviation in y-direction. Ideally, the density of the fluid at the point of detection should be lower than the density of the measured particles, otherwise the particles in the sample will by captured in the density gradient and not reach the detector. Although only a single measurement was performed for the estimation of the virus particle density in two different fluids, a good approximation was observed. The correctness of the proposed model, based on Stokes and centrifugal force, was evaluated by triplicate measurements of polyvinyl chloride (PVC) and silicon (IV) oxide particles in 4 different gradients. High correlation factors of 0.9993 (PVC, FIG. 28) and 0.9883 (silicon oxide, data not shown) confirmed the linear relation between the used parameters, thus verifying the proposed model. Although only small density differences (the regression based x-value) but high viscosity differences by a factor of up to 6.5 (by dividing equation 8 for the highest by the lowest viscous fluid with the standard particle measurement) were used, standard deviation was less than 0.9% and 1.2% for PVC and silicon (IV) oxide particles, respectively.

For all applied particles, density determination by the proposed method resulted in good approximations between determined and reference particle density with a difference of maximum 15%. For virus particles, the use of only two measuring fluids (containing 2-8% (w/v) and 14-20% (w/v) sucrose) allowed the density determination with a difference of less than 4%, due to the small difference in particle and fluid densities. Obviously, the conditions used are appropriate to enable estimation of particle densities with high precision. Furthermore, use of equation (7) allowed robust and highly reproducible calculation of the settling velocity. Triplicate measurement in 4 different density gradients with a varying density of only 20% but a varying dynamic viscosity of 650% showed a standard deviation of each less than 1.2%. The correlation coefficient of more than 0.98 for the linear regression together with the high reproducibility indicated high robustness as well as a good model fit. Also, the use of additional methods for particle quantification, such as virus titration, electron microscopy or ELISA can be avoided, which, if available, are time consuming (in the case of virus titration or electron microscopy, up to several days) and error prone. For the measured virus particles, a density determination using two different fluids was (dependent on the size of virus particles) possible in less than one hour. Therefore, this method could, for example, be used for the particle density determination for unknown viruses. The determined virus particle density could then be used to support the design and optimization of downstream processing steps, e.g. density gradient centrifugation with isopycnic fractionation.

Additional measurements, or an additional fluid, could further improve the estimation of particle densities.

Evaluation of Microbial Contamination in a Sample

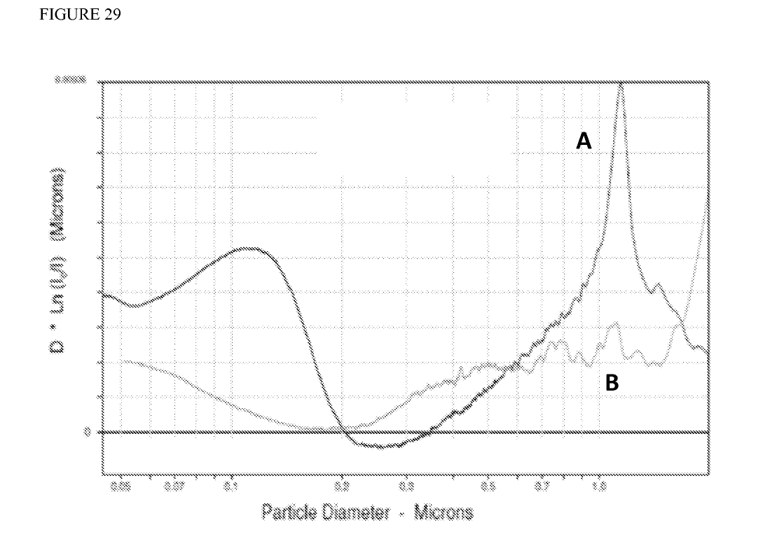

The inventors also tested whether disc centrifugation may be used to identify the presence of microbes in a sample. Microbial particles are typically 1 μm or more in diameter, and are thus much larger than most virus particles. Accordingly, their respective peaks may be easily distinguished from each other in a particle size distribution comprising virus and microbial particles. FIG. 29 (curve A) shows a distinct peak at approximately 1.1 μm, which corresponds to microbial particles in the sample tested.

The inventors then performed an aseptic filtration step on the contaminated sample, to determine whether this aseptic filtration step was effective at removing microbial contamination. FIG. 29 (curve B) shows that the aseptic filtration step effectively removed the microbial contaminant from the sample, as shown by the significant reduction in the peak at approximately 1.1 μm.

These results demonstrate that the invention is useful for identifying microbial contamination in a sample, and also for determining whether an aseptic filtration step has been successful.

Evaluation of Splitting

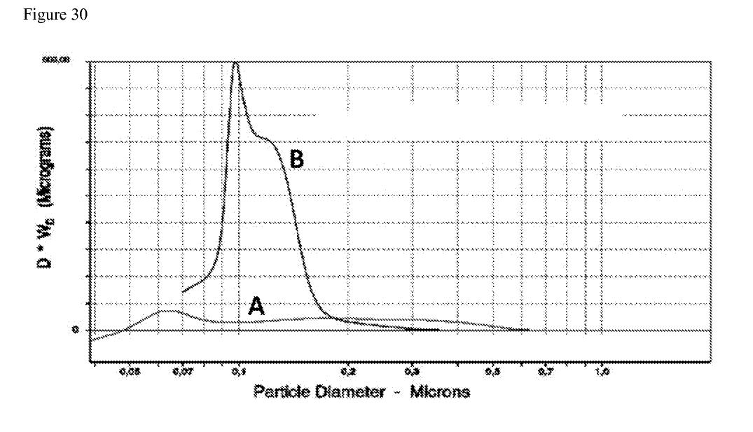

The inventors also tested whether disc centrifugation may be used to determine whether administration of a splitting agent could successfully split the biological particles, and by how much. In this case, the biological particles were virus particles. FIG. 30 clearly shows the particle size distribution for a sample containing influenza particles (curve B). FIG. 30 (curve A) shows the particle size distribution for the same sample, after virus splitting. Successful virus splitting is clearly evident by the effective removal of the peak corresponding to the virus particles.

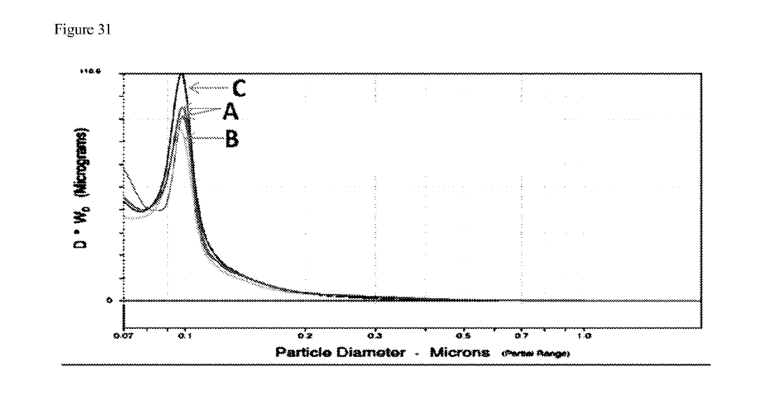

To test whether disc centrifugation may be used to identify incomplete virus splitting, the inventors treated the virus sample with less splitting agent (i.e. FIG. 31, curves A and B). FIG. 31, curves A and B (treatment curves) are similar to control curve C, demonstrating that treatment with less splitting agent did not result in complete splitting. Even though the amount of splitting agent used did not split substantially all of the virus particles (e.g. as shown in FIG. 30, curve A), it can be seen that the maximum weight of the size distribution of curves A and B is less than that of curve C, thereby indicating that the amount of whole virus particles has decreased slightly (i.e. the amount of split virus particles has increased).

These results demonstrate that methods of the invention are useful for identifying whether a sample (e.g. a virus sample) comprises split (virus) particles, particularly after treatment with a splitting agent. This approach may also be used for determining the effectiveness of a splitting agent, and also the susceptibility of a biological sample to splitting. This approach may also be used to identify the optimal amount of splitting agent for use in a particle splitting protocol.

Evaluation of Adjuvant Particles

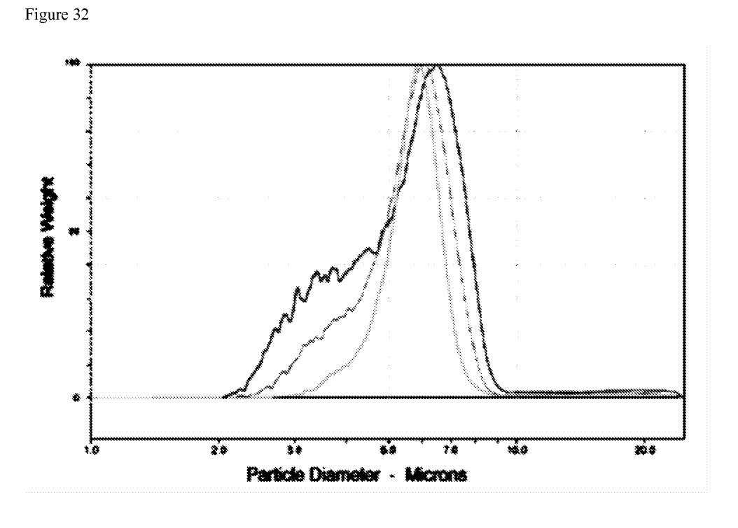

The inventors also tested whether disc centrifugation may be used to reproducibly quantify inorganic particles, such as adjuvant particles. FIG. 32 shows triplicate measurements of the particle size distribution for two different adjuvant particles (aluminium hydroxide and aluminium phosphate), each of which has an approximate hydrodynamic diameter of 6 μm. FIG. 32 shows that disc centrifugation can accurately and reproducibly quantify adjuvant particles. These data also show that disc centrifugation is also suitable for analysing particles with a larger hydrodynamic diameter (in this case, approximately 6 μm).

### CONCLUSION

The inventors found that disc centrifugation also provides a fast method for particle size determination that is based on particle settling velocity and photometric detection (see reference 46). Compared to other methods in the prior art, e.g. dynamic light scattering, disc centrifugation provides high resolution results (see references 47 and 48). This is due e.g. to the application of standards which render the measurement of the fluid rheological properties reproducible and precise.

Furthermore, parameters such as viscosity, which are difficult to estimate for the measuring process, do not have do be known by the user. The most critical factor for an absolute correct size of particles is the particle density. Previously, users were dependent on data in the literature, or external particle density measurements. Here, the inventors provide a model for the estimation of particle density. By applying this method of the invention, the inventors demonstrated that disc centrifugation alone can be used to determine absolute particle size, thereby eliminating the reliance on the literature and numerous external measurements, and techniques. The only externally measured parameters that are required are the densities of the at least two separating fluids, which may be easily derived using methods that are well known in the prior art.

Furthermore, the inventors found that this method of the invention can be improved by using two particle standards with different densities, thus completely eliminating the need for any externally measures parameters, including fluid density.

The method of the invention is particularly useful for determining the density and size of virus particles. Analysis of virus particles with known densities revealed that the invention is capable of accurately estimating the density of virus particles. Previous measurements of particle density are associated with a number of problems, as outlined above. The present invention provides a method for particle density estimation that is fast, accurate and reproducible, and independent of external measurements. This, the method of the invention allows particle quantification using as few as two measurements, obtained by disc centrifugation, and requires only a small subset of easily measurable parameters.

### REFERENCES

[1] WO2010/128100
[2] Colard et al. (2010). Langmuir 26(11): 7915-7921
[3] U.S. Pat. No. 5,786,898
[4] Kistner et al. (1998) Vaccine 16:960-8.
[5] Kistner et al. (1999) Dev Biol Stand 98:101-10.
[6] Bruhl et al. (2000) Vaccine 19:1149-58.
[7] WO97/37000.
[8] Brands et al. (1999) Dev Biol Stand 98:93-100.
[9] Halperin et al. (2002) Vaccine 20:1240-7.
[10] Tree et al. (2001) Vaccine 19:3444-50.
[11] Kistner et al. (1998) Vaccine 16:960-8.
[12] Kistner et al. (1999) Dev Biol Stand 98:101-110.
[13] Bruhl et al. (2000) Vaccine 19:1149-58.
[14] Pau et al. (2001) Vaccine 19:2716-21.
[15] http://www.atcc.org/
[16] http://locus.umdnj.edu/
[17] WO03/076601.
[18] WO2005/042728.
[19] WO03/043415.
[20] WO01/85938.
[21] WO2006/108846.
[22] WO97/37000.
[23] Brands et al. (1999) Dev Biol Stand 98:93-100.
[24] Halperin et al. (2002) Vaccine 20:1240-7.
[25] EP-A-1260581 (WO01/64846).
[26] WO2006/071563.
[27] WO2005/113758.
[28] WO03/023021
[29] WO03/023025
[30] Vaccines. (eds. Plotkin & Orenstein). 4th edition, 2004, ISBN: 0-7216-9688-0.
[31] WO02/28422.
[32] WO02/067983.
[33] WO02/074336.
[34] WO01/21151.
[35] WO02/097072.
[36] WO2005/113756.
[37] Huckriede et al. (2003) Methods Enzymol 373:74-91.
[38] Remington: The Science and Practice of Pharmacy (Gennaro, 2000; 20th edition, ISBN: 0683306472)
[39] Vaccine Design: The Subunit and Adjuvant Approach (eds. Powell & Newman) Plenum Press 1995 (ISBN 0-306-44867-X).
[40] Vaccine Adjuvants: Preparation Methods and Research Protocols (Volume 42 of Methods in Molecular Medicine series). ISBN: 1-59259-083-7. Ed. O'Hagan.
[41] Vaccine Design: The Subunit and Adjuvant Approach (eds. Powell & Newman) Plenum Press 1995 (ISBN 0-306-44867-X).
[42] Wood J. M. et al. (1977) Journal of Biological Standardization, 5(3), pp. 237-247
[43] Campbell et al. (2004) Biomacromolecules (5): 1728-1735
[44] Heim (2004). Part. Part. Syst. Char.
[45] The Springer Index of Viruses. Tidona, C. A. & Darai, G. (2001) Berlin, Heidelberg, New York.
[46] Bondoc et al. (1998). J. Ind. Microbiol, 20: 317-322
[47] Mahl et al. (2010). Colloid Surface A.
[48] Couteau & Roebben. (2007) Proceedings of the EuroNanoForum, Nanotechnology in Industrial Applications p. 66-68

1. A method for quantifying virus particles in a sample using a disc centrifuge, wherein the concentration of virus particles in the sample is unknown.

2. The method of claim 1, wherein the virus sample comprises non-aggregated and/or aggregated virus particles.

3. The method of claim 2, comprising the steps of:
a. separating particles in the sample by disc centrifugation;
b. detecting the particles using a particle detector;
c. measuring the particle size distribution;
d. identifying the presence or absence of:
i. non-aggregated virus particles, based on the presence of a unimodal size distribution; and/or
ii. aggregated virus particles, based on the presence of a multimodal size distribution;
e. determining:
i. the maximum weight of the size distribution and/or the integrated weight of the size distribution for a sample comprising non-aggregated virus particles; or
ii. the integrated weight of the size distribution for a sample comprising aggregated virus particles;
f. comparing (i) the maximum weight of the size distribution for the sample with the maximum weight of the size distribution of a reference, and/or (ii) comparing the integrated weight of the size distribution for the sample with integrated weight of the size distribution of a reference, and thereby quantifying the virus particles in the sample.

4. The method according to claim 3, wherein the particles are separated in step (a) using a density gradient.

5. The method according to claim 4, wherein the density gradient further comprises:
a) a salt comprising sodium chloride, or
b) a buffer comprising sodium phosphate.

6. The method according to claim 5, wherein the pH of the separating fluid is between pH3 and 11.

7. (canceled)

8. (canceled)

9. (canceled)

10. (canceled)

11. (canceled)

12. (canceled)

13. (canceled)

14. The method according to claim 3 further comprising detecting the presence or absence of contaminating virus particles in a sample.

15. (canceled)

16. (canceled)

17. (canceled)

18. (canceled)

19. (canceled)

20. (canceled)

21. (canceled)

22. The method according to claim 3 wherein the virus particles comprise adjuvant particles and antigen and further comprises the steps of:
(a) introducing to the sample compound(s) that (i) bind adjuvant particles, (ii) do not bind soluble antigen, and (iii) comprise a detectable label;
(b) identifying the presence or absence of particle size distribution(s) corresponding to
(i) adjuvant particles that are not adsorbed to antigen,
and/or
(ii) adjuvant particles to which antigen is adsorbed.

23. (canceled)

24. The method according to claim 3 wherein the virus particles comprise adjuvant particles and antigen and further comprising the steps of:
(a) introducing to the sample compound(s) that (i) bind antigen, (ii) do not bind adjuvant, and (iii) comprise a detectable label;
(b) identifying the presence or absence of particle size distribution(s) corresponding to
(i) antigen not adsorbed to adjuvant,
and/or
(iii) antigen adsorbed to adjuvant particles.

25. The method of claim 24, wherein the compound(s) that (i) bind antigen, (ii) do not bind adjuvant, and (iii) comprise a detectable label is protein-specific and/or nucleic acid-specific.

26. (canceled)

27. (canceled)

28. A process for manufacturing a vaccine comprising the steps of:
(i) quantifying non-aggregated and/or aggregated virus particles in a sample taken from a bulk material by using the method according to claim 3;
(ii) optionally adjusting the concentration of the virus particles in the bulk material, based on the quantity of virus particles in the sample, to a concentration that is suitable for use in a vaccine composition; and
(iii) preparing the vaccine from the bulk material.

29. A method for determining particle density and/or size using a disc centrifuge comprising the steps of:
a. measuring the settling velocity of a sample comprising an unknown concentration of virus particles in at least two different separating fluids, wherein the fluids have different rheological properties;
b. performing a regression analysis, preferably a linear regression analysis.

30. The method according to claim 29 further comprising determining particle sedimentation velocity comprising:
a. measuring the distance from the settling starting point to the detector;
b. determining the retention time;
c. dividing the value obtained from step (a) by the value obtained from step (b).

31. (canceled)

32. (canceled)

33. (canceled)

34. (canceled)

35. A method of identifying abnormal particle size, density and/or quantity in a biological sample using a disc centrifuge comprising the steps of:
a. measuring the particle size distribution of a biological sample, wherein the concentration of the particles in the biological sample is unknown;
b. comparing the particle size distribution of the biological sample with the particle size distribution of a control;
c. identifying whether the particle size distribution of the particles in the biological sample differs from the particle size distribution of the control sample;
d. determining whether the biological sample contains an abnormally high, low or normal level of particles, compared to the control.

36. (canceled)

37. (canceled)

38. A vaccine composition produced by the method according to claim 3.
